(19) United States
(12) Patent Application Publication
(10) Pub. No.: US 2025/0122532 A1
(43) Pub. Date: Apr. 17, 2025

(54) VIRAL VECTORS AND PACKAGING CELL LINES

(71) Applicant: Umoja Biopharma, Inc., Seattle, WA (US)

(72) Inventors: Andrew SCHARENBERG, Seattle, WA (US); Laurie BEITZ, Seattle, WA (US)

(21) Appl. No.: 18/991,101

(22) Filed: Dec. 20, 2024

Related U.S. Application Data
(63) Continuation of application No. 17/046,701, filed on Oct. 9, 2020, now Pat. No. 12,215,337.
(60) Provisional application No. 62/656,823, filed on Apr. 12, 2018.

(51) Int. Cl. C12N 15/86; A61K 9/00; A61K 31/436; A61K 38/13; A61K 38/17; A61K 39/395; C07K 14/55; C07K 14/705; C07K 16/28

(52) U.S. Cl. CPC C12N 15/86; A61K 9/0019; A61K 9/0053; A61K 31/436; A61K 38/13; A61K 38/177; A61K 38/1774; A61K 38/1793; A61K 39/3955; C07K 14/55; C07K 14/70532; C07K 14/70578; C07K 16/2809; C07K 2317/622; C07K 2317/76; C07K 2319/02; C07K 2319/30; C12N 2740/15032; C12N 2740/15041; C12N 2740/15043; C12N 2740/15052; C12N 2740/16032; C12N 2740/16041; C12N 2740/16052

(57) ABSTRACT

The disclosure relates generally to nucleic acid vectors and packaging cell lines for in vivo expansion of T-cells. More particularly, the disclosure relates to direct intratumoral injection of a lentiviral vector adapted for transduction and drug-mediated expansion of tumor-infiltrating lymphocytes in vivo.

### CROSS-REFERENCE TO RELATED APPLICATIONS

The present application is a continuation of U.S. National Phase application Ser. No. 17/046,701, filed Oct. 9, 2020, based on International Application No. PCT/US2019/026923, filed Apr. 11, 2019, which claims priority under 35 U.S.C. § 119 from Provisional Application Ser. No. 62/656,823, filed Apr. 12, 2018, the disclosures of which are incorporated herein by reference including any drawings.

### DESCRIPTION OF THE TEXT FILE SUBMITTED ELECTRONICALLY

The Sequence Listing associated with this application is provided in text format in lieu of a paper copy, and is hereby incorporated by reference into the specification. The name of the text file containing the Sequence Listing is 2024 Nov. 13 Sequence_Listing_ST26 061479-501C01US.xml. Tis file is 95,571 bytes, was created on Nov. 13, 2024, and is being submitted electronically via EFS-Web.

### FIELD OF THE INVENTION

The disclosure relates generally to viral vectors, packaging cell lines, and related methods of use and in particular for expansion of immune cell populations in vivo for treatment of a disease condition.

### BACKGROUND OF THE INVENTION

Cancer immunotherapy is a treatment modality based on therapeutic induction of immune responses to tumors. Adoptive T cell therapy (ACT) is one form of cancer immunotherapy. Lymphocytes, particularly tumor-infiltrating lymphocytes (TILs), are isolated from the body, cultured ex vivo, expanded, and then re-infused. The expansion step may include antigen-specific expansion or genetic engineering of the TILs. ACT is reviewed in Rosenberg et al. Adoptive cell transfer as personalized immunotherapy for human cancer. Science. 348:62-8 (2015).

The present inventors have realized that, as an alternative to ACT, in vivo transduction of TILs, or other immune cells, could potentiate expansion of cells in vivo rather than ex vivo. Furthermore, the present inventors have realized in vivo transduction of TILs, or other immune cells, would allow for treatment of cancer or other disease conditions without the costly, time-consuming, and risky procedures required by ACT.

Accordingly, there is a need for means of expanding populations of TILs or other immune cells in vivo. In particular, there is a need for therapeutic agents capable of selectively expanding desirable populations of TILs or other immune cells in vivo. The present disclosure provides viral vectors, packaging cell lines, and related methods of use for expansion of TILs or other immune cells in vivo for treatment of a disease condition.

### SUMMARY OF THE INVENTION

The present disclosure is based, in part, on the discovery that lentiviral vectors and packaging cell lines designed to express T-cell/NK-cell activation receptors (as well as optionally other effector proteins) are useful for in vivo expansion of T-cells. Lentiviral vectors and packaging cell lines of the present disclosure may be adapted for transduction and drug-mediated expansion of tumor-infiltrating lymphocytes in vivo, such as when the lentiviral vector is packaged into a lentiviral particle using a packaging cell line and the resulting lentiviral particles are delivered into the body of a subject. Intratumoral injection of lentiviral particles according to the present disclosure results in in vivo expansion of tumor-infiltrating lymphocytes. Expansion of tumor-infiltrating lymphocytes is controllable by use of a small molecule, when the T-cell/NK-cell activation receptor is capable of being activated (or inactivated) by such a small molecule. Optionally, the packaging cell line expresses a T-cell activation or co-stimulation molecule, facilitating T-cell transduction by lentiviral particles derived from the packaging cell line in the absence of exogenous activating agents. Optionally, the lentiviral vector confers resistance to immunosuppressive drug(s), facilitating selective expansion of target cells.

In one aspect, the disclosure provides a nucleic acid vector comprising a T-cell and/or NK-cell specific promoter operatively linked to a nucleic acid sequence encoding T-cell/NK-cell activation receptor, wherein the T-cell/NK-cell activation receptor is capable of being activated by a small molecule. In an embodiment, the nucleic acid vector is a lentiviral vector. In an embodiment, the nucleic acid vector comprises a sequence at least partially identical to SEQ ID NOs: 6-11 or fragments thereof.

In another aspect, the disclosure provides a nucleic acid vector comprising a strong promoter operatively linked to a nucleic acid sequence encoding a T-cell/NK-cell activation receptor, wherein the T-cell/NK-cell activation receptor is capable of being activated by a small molecule. In an embodiment, the strong promoter is selected from SEQ ID NOs: 1-5.

In another aspect, the disclosure provides a packaging cell line for generating lentiviral particles capable of activating and efficiently transducing T cells, comprising cultured cells capable of packaging a lentivirus vector, wherein the cultured cells are genetically engineered to express a T-cell activation or co-stimulation molecule.

In another aspect, the disclosure provides a lentiviral particle comprising a nucleic acid vector, such as any of the nucleic acid vectors of the present disclosure. In an embodiment, the lentiviral particle comprises a T-cell activation or co-stimulation molecule, e.g., an anti-CD3 antibody, CD28 ligand, or 41bb ligand.

In another aspect, the disclosure provides a lentiviral particle for activating and efficiently transducing T cells, prepared by transducing the nucleic acid vector of claim 19 into cultured cells genetically engineered to express a T-cell activation or co-stimulation molecule.

In another aspect, the disclosure provides a method for treating a subject suffering from cancer, comprising administering any of the lentiviral particles of the present disclosure to the subject, and administering a small molecule (the T-cell/NK-cell activation receptor of the lentiviral particle being capable of being activated by the small molecule) to the subject, wherein the cancer is treated in the subject.

In another aspect, the disclosure provides a method for expanding T-cells capable of recognizing and killing tumor cells in a subject in need thereof, comprising administering any of the lentiviral particles of the present disclosure to the subject, and administering a small molecule (the T-cell/NK-cell activation receptor of the lentiviral particle being capable of being activated by a small molecule) to the subject, wherein T-cells capable of recognizing and killing tumor cells in the subject are expanded.

In another aspect, the disclosure provides a nucleic acid comprising a promoter specific to T-cells, NK cells, or T-cells and NK cells, wherein the sequence of the nucleic acid is at least 90% identical to a sequence selected from the group consisting of SEQ ID NOs: 1-4.

Additional aspects and embodiments of the disclosure will be apparent from the detailed description that follows.

### BRIEF DESCRIPTION OF THE DRAWINGS

FIGS. 1A-1B depict lentiviral particles. FIG. 1A is a diagram of an embodiment of the surface-engineered lentiviral particle, which comprises surface-expressed anti-CD3 and T-cell co-stimulatory molecules not present in the lentiviral particle depicted in FIG. 1B.

FIG. 2 depicts the experimental protocol used to generate HATSE-293 packaging cell line from HEK-293T cells transduced with a lentiviral vector encoding anti-CD3 scFV, CD86, and CD137L. CD86⁺CD137L⁺were isolated by fluorescence-activated cell sorting, expanded, and frozen for long-term storage and use.

FIGS. 3A-3K depict FACS analysis of parental 293 cells and the HATSE-293packaging cell line for CD46 (expected constitutive expression on both cell lines), and CD86^{+/}CD137L⁺ expression (expression expected only in the HATSE-293 cell line).

FIGS. 3A-3E depict analysis of the parental HEK-293 cell line for CD46 (expected constitutive expression), and CD86/CD137L expression (no expression expected). No detectable fluorescence labeling was observed in the unstained (mock) samples (FIG. 3A). High expression of CD46 was detected by anti-CD46 antibody staining (FIG. 3A-B; PE anti-human CD46), and no expression of either CD86 (FIG. 3E; pacific blue anti-human CD86) or CD137L (FIG. 3D; PE anti-human 4-1BB ligand (CD137L)) detected by specific staining for CD86 and CD137L, respectively.

FIGS. 3F-3K depict analysis of the HATSE-293 cell line for for CD46 (expected constitutive expression on both cell lines), and CD86/CD137L expression (CD86⁺/CD137L⁺ expression expected). FIG. 3F and FIG. 3G demonstrate no detectable fluorescence labeling in the unstained (mock) samples. FIG. 3H and FIG. 3I demonstrate uniform high expression of CD46 detected by anti-CD46 antibody staining (PE anti-human CD46). FIG. 3J and FIG. 3K demonstrate two populations that express low and high amounts of either CD86 or CD137L are detected using anti-CD86 (FIG. 3J) and anti-CD137L (FIG. 3K) antibodies, respectively.

FIG. 4 depicts growth curves of T-cells transduced by control lentiviral particles or lentiviral particles generated from the HATSE cell lines described in the definitions and examples that follow. As a positive control, T-cells were transduced with control lentiviral particles in the presence of stimbeads. “Days post transduction” refers to the number of days elapsed after the day on which the cells were exposed to particles (day 0).

FIG. 5 depicts fluorescence micrographs of cells exposed to 293T control particles [293T no stim]; 293T control particles and stimbeads [293T stim]; 1× sorted HATSE particles [1× sorted HATSE (no stim)]; or 2× sorted HATSE particles [2× sorted HATSE (no stim)].

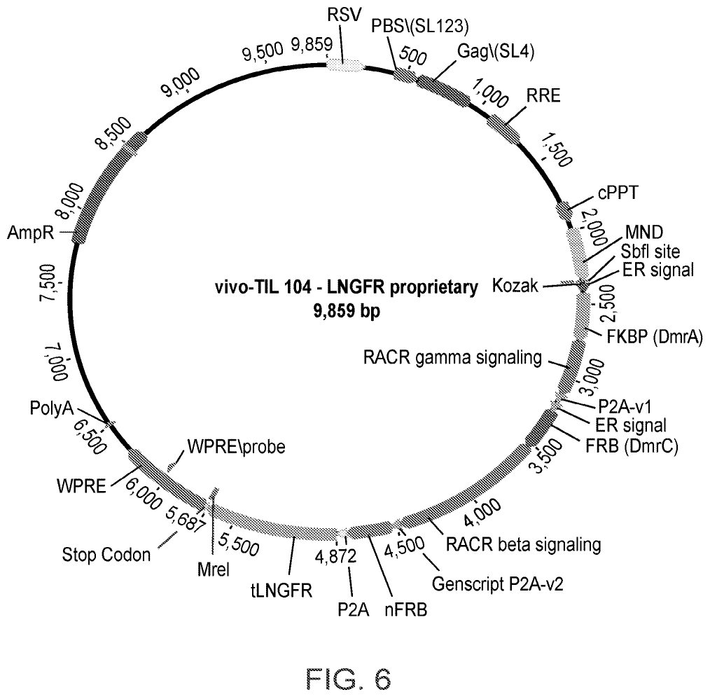

FIG. 6 depicts a vector map for vivo-TIL 104 LNGFR (SEQ ID NO: 6).

FIG. 7 depicts a vector map for vivo-TIL 105 TCP1 (SEQ ID NO: 7).

FIG. 8 depicts a vector map for vivo-TIL 106 TCP2 (SEQ ID NO: 8).

FIG. 9 depicts a vector map for vivo-TIL 107 TCP3 (SEQ ID NO: 9).

FIG. 10 depicts a vector map for vivo-TIL 108 TCP4 (SEQ ID NO: 10).

FIG. 11 depicts a vector map for vivo-TIL 109 hPerfP (SEQ ID NO: 11).

FIGS. 12A-12F show results for an exemplary small-molecule controllable T-cell/NK-cell activation receptor, termed RACCR. FIG. 12A illustrates the design the exemplified RACCR molecule. FIG. 12A shows cells transduced with a control lentiviral particle expressing GFP. FIG. 12B shows cells transduced with a surface-engineered particle expressing mCherry. FIG. 12C shows cells transduced with a surface-engineered particle expressing RACCR.

FIG. 12E shows a graph of T-cell expansion of T cells transduced with various surface engineered lentiviral particles (SE-LVP), including SE-LVPs expressing RACCR, in the presence of IL-2 or rapamycin. FIG. 12F shows data confirming T cell expansion driven by RACCR is controllable with rapamycin.

### DETAILED DESCRIPTION

The present inventors have realized that, as an alternative to ACT, in vivo transduction of TILs, or other immune cells, could potentiate expansion of cells in vivo rather than ex vivo. Furthermore, the present inventors have realized in vivo transduction of TILs, or other immune cells, allows for treatment of cancer or other disease conditions without the drawbacks of ex vivo expansion of immune cells.

Thus, the present disclosure provides means of expanding populations of TILs or other immune cells in vivo. In particular, the present disclosure provides therapeutic agents capable of selectively expanding desirable populations of TILs or other immune cells in vivo. The present disclosure provides viral vectors and related methods of use for expansion of TILs or other immune cells in vivo for treatment of a disease condition.

The present disclosure is based, in part, on the discovery that lentiviral vectors and packaging cell lines designed to express T-cell/NK-cell activation receptors (as well as optionally other effector proteins) are useful for in vivo expansion of T-cells. Lentiviral vectors and packaging cell lines of the present disclosure may be adapted for transduction and drug-mediated expansion of tumor-infiltrating lymphocytes in vivo, such as when the lentiviral vector is packaged into a lentiviral particle using a packaging cell line and the resulting lentiviral particles are delivered into the body of a subject, such as to a patient suffering from a solid tumor. The lentiviral vectors and particles disclosed can be used for ACT by in vitro transduction of autologous or allogeneic T cells or other immune cells. In some cases, the lentiviral vectors and particles disclosure are configured for use in vivo. Intratumoral injection of lentiviral particles results in in vivo expansion of tumor-infiltrating lymphocytes because the lentiviral vectors are configured to provide expression of T-cell/NK-cell activation receptors capable of providing mitogenic signals to in vitro or in vivo transduced target cells.

### 1.1 Nucleic Acid Vectors

As used herein, the term “nucleic acid vector” is intended to mean any nucleic acid that functions to carry, harbor, or express a nucleic acid of interest. Nucleic acid vectors can have specialized functions such as expression, packaging, pseudotyping, or transduction. Nucleic acid vectors also can have manipulatory functions if adapted for use as a cloning or shuttle vector. The structure of the vector can include any desired form that is feasible to make and desirable for a particular use. Such forms include, for example, circular forms such as plasmids and phagemids, as well as linear or branched forms. A nucleic acid vector can be composed of, for example, DNA or RNA, as well as contain partially or fully, nucleotide derivatives, analogs and mimetics. Such nucleic acid vectors can be obtained from natural sources, produced recombinantly or chemically synthesized.

Non-limiting examples of vector systems of the present disclosure include a retrovirus, a lentivius, a foamy virus, and a Sleeping Beauty transposon.

### 1.1.1 Lentiviral Vectors

Lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses (HIV-1 and HIV-2) and the Simian Immunodeficiency Virus (SIV). Lentiviral vectors have been generated by multiply attenuating the HIV virulence genes, for example, the genes env, vif, vpr, vpu and nef are deleted, making the vector biologically safe.

Lentiviral vectors offer great advantages for gene therapy. Unless engineered to be non-integrating, lentiviral vectors integrate stably into chromosomes of target cells, permitting long-term expression of delivered transgenes. Further, they do not transfer viral genes therefore avoiding the problem of generating transduced cells that can be destroyed by cytotoxic T-cells. Furthermore, they have a relatively large cloning capacity, sufficient for most envisioned clinical applications. In addition, lentiviruses, in contrast to other retroviruses, are capable of transducing non-dividing cells. This is very important in the context of gene-therapy for tissues such as the hematopoietic system, the brain, liver, lungs and muscle. For example, vectors derived from HIV-1 allow efficient in vivo and ex vivo delivery, integration and stable expression of transgenes into cells such a neurons, hepatocytes, and myocytes (Blomer et al., 1997; Kafri et al., 1997; Naldini et al., 1996; Naldini et al., 1998).

Lentiviral vectors are known in the art, see Naldini et al. (1996) Science 272:263-7; Zuffercy et al. (1998) J. Virol. 72:9873-9880; Dull et al. (1998) J. Virol. 72:8463-8471; U.S. Pat. Nos. 6,013,516; and 5,994,136, which are each incorporated herein by reference in its entirety. In general, these vectors are configured to carry the essential sequences for selection of cells containing the vector, for incorporating foreign nucleic acid into a lentiviral particle, and for transfer of the nucleic acid into a target cell.

A commonly used lentiviral vector system is the so-called third-generation system. Third-generation lenviral vector systems include four plasmids. The “transfer plasmid” encodes the polynucleotide sequence that is delivered by the lentiviral vector system to the target cell. The transfer plasmid generally has one or more transgene sequences of interest flanked by long terminal repeat (LTR) sequences, which facilitate integration of the transfer plasmid sequences into the host genome. For safety reasons, transfer plasmids are generally designed to make the resulting vector replication incompetent. For example, the transfer plasmid lacks gene elements necessary for generation of infective particles in the host cell. In addition, the transfer plasmid may be designed with a deletion of the 3′ LTR, rendering the virus “self-inactivating” (SIN). See Dull et al. (1998) J. Virol. 72:8463-71; Miyoshi et al. (1998) J. Virol. 72:8150-57.

Third-generation systems also generally include two “packaging plasmids” and an “envelope plasmid.” The “envelope plasmid” generally encodes an Env gene operatively linked to a promoter. In an exemplary third-generation system, the Env gene is VSV-G and the promoter is the CMV promoter. The third-generation system uses two packaging plasmids, one encoding gag and pol and the other encoding rev as a further safety feature—an improvement over the single packaging plasmid of so-called second-generation systems. Although safer, the third-generation system can be more cumbersome to use and result in lower viral titers due to the addition of an additional plasmid. Exemplary packing plasmids include, without limitation, pMD2.G, pRSV-rev, pMDLG-pRRE, and pRRL-GOI.

Lentiviral vector systems rely on the use of a “packaging cell line.” In general, the packaging cell line is a cell line whose cells are capable of producing infectious lentiviral particles when the transfer plasmid, packaging plasmid(s), and envelope plasmid are introduced into the cells. Various methods of introducing the plasmids into the cells may be used, including transfection or electroporation. In some cases, a packaging cell line is adapted for high-efficiency packaging of a lentiviral vector system into lentiviral particles.

As used herein, the term “lentiviral vector” is intended to mean a nucleic acid that encodes a lentiviral cis nucleic acid sequence required for genome packaging. A lentiviral vector also can encode other cis nucleic acid sequences beneficial for gene delivery, including for example, cis sequences required for reverse transcription, proviral integration or genome transcription. A lentiviral vector performs transduction functions of a lentiviral vector. As such, the exact makeup of a vector genome will depend on the genetic material desired to be introduced into a target cell. Therefore, a vector genome can encode, for example, additional polypeptides or functions other than that required for packaging, reverse transcription, integration, or transcription. Such functions generally include coding for cis elements required for expression of a nucleic acid of interest. The lentiviral cis sequences or elements can be derived from a lentivirus genome or other virus or vector genome so long as the lentiviral vector genome can be packaged by a packaging cell line into a lentiviral particle and introduced into a target cell.

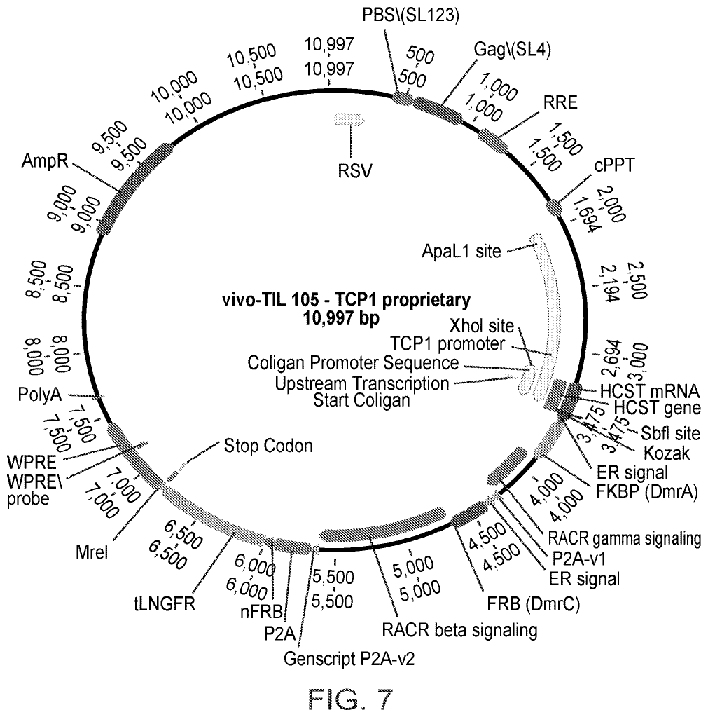

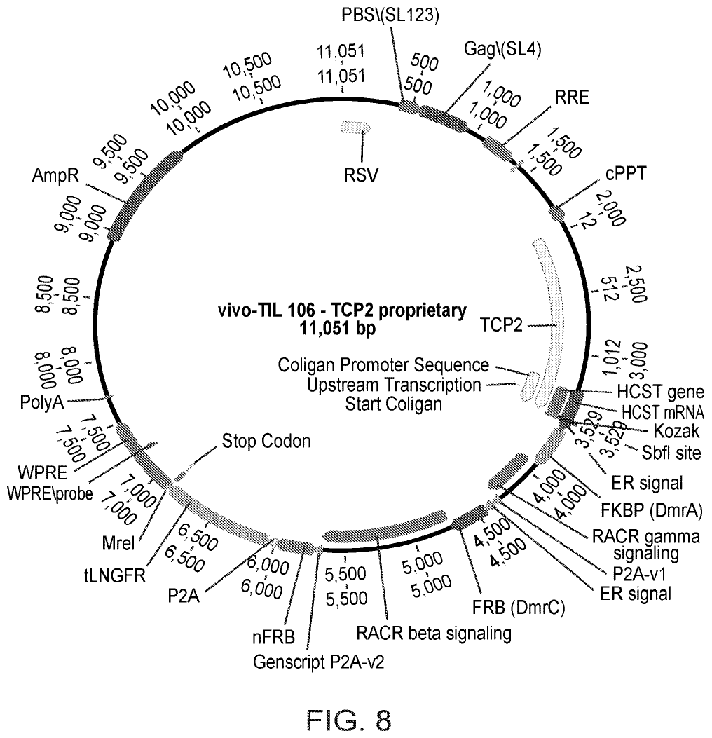

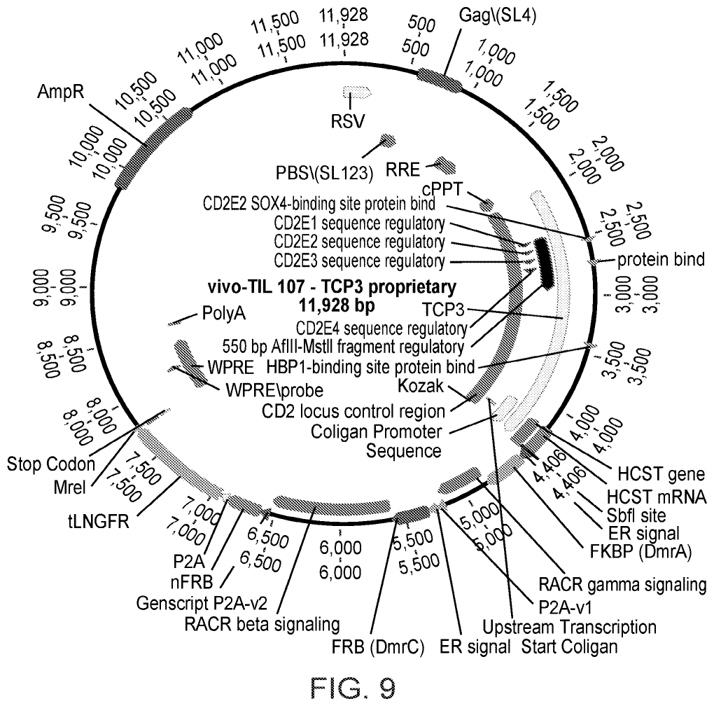

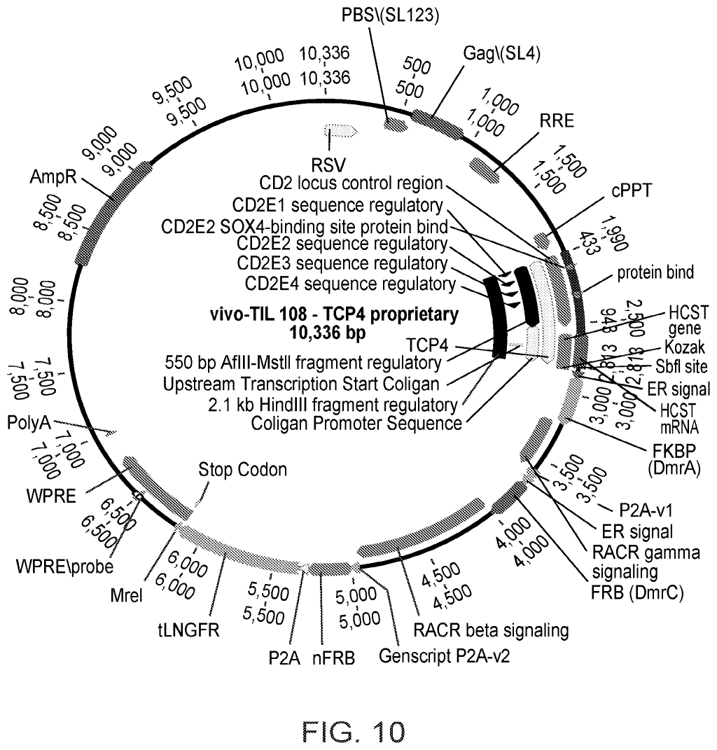

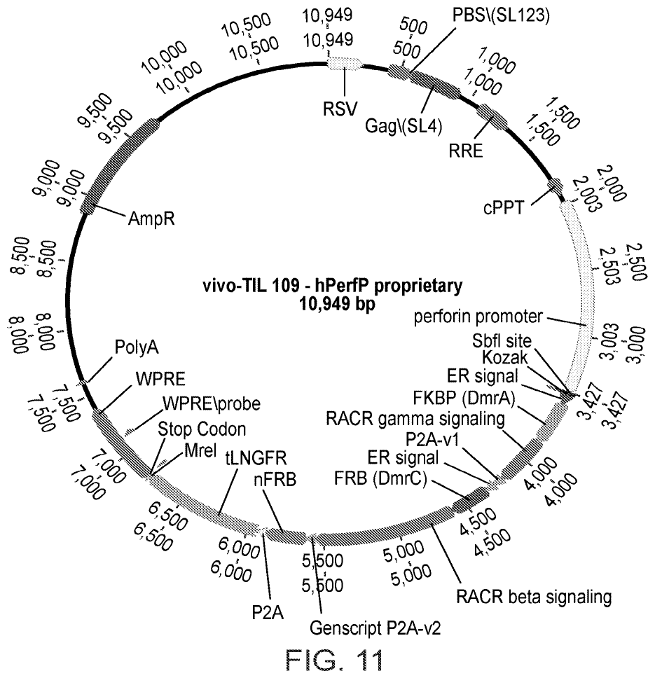

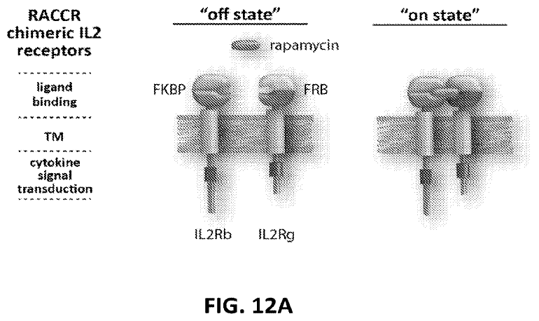

Non-limiting examples of lentiviral vectors include SEQ ID NOs: 6-11, which are illustrated in FIGS. 6-11.

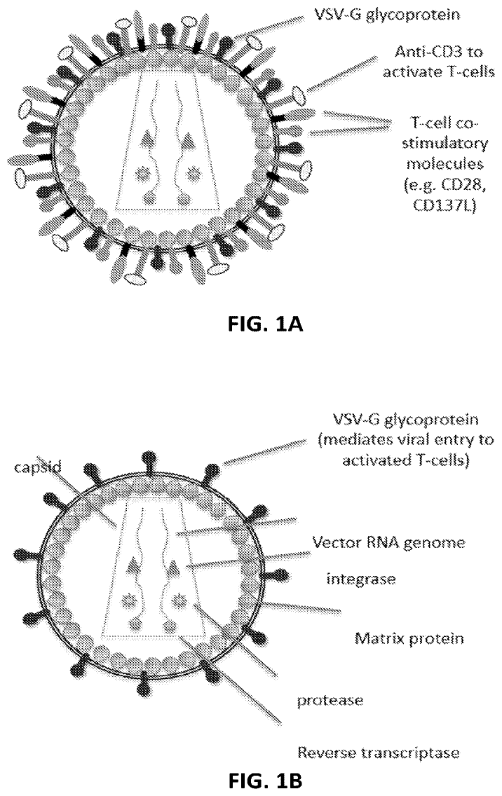

The lentiviral particles produced generally include an RNA genome (derived from the transfer plasmid), a lipid-bilayer envelope in which the Env protein is embedded, and other accessory proteins including integrase, protease, and matrix protein (see FIG. 1B). As used herein, the term “lentiviral particle” is intended to mean a viral particle that includes an envelope, has one or more characteristics of a lentivirus, and is capable of invading a target host cell. Such characteristics include, for example, infecting non-dividing host cells, transducing non-dividing host cells, infecting or transducing host immune cells, containing a lentiviral virion including one or more of the gag structural polypeptides p7, p24, and p17, containing a lentiviral envelope including one or more of the env encoded glycoproteins p41, p120, and p160, containing a genome including one or more lentivirus cis-acting sequences functioning in replication, proviral integration or transcription, containing a genome encoding a lentiviral protease, reverse transcriptase or integrase, or containing a genome encoding regulatory activities such as Tat or Rev. The transfer plasmids may comprise a cPPT sequence, as described in U.S. Pat. No. 8,093,042.

The efficiency of the system is an important concern in vector engineering. The efficiency of a lentiviral vector system may be assessed in various ways known in the art, including measurement of vector copy number (VCN) or vector genomes (vg) such as by quantitative polymerase chain reaction (qPCR), or titer of the virus in infectious units per milliliter (IU/mL). For example, the titer may be assessed using a functional assay performed on the cultured tumor cell line HT1080 as described in Humbert et al. Development of Third-generation Cocal Envelope Producer Cell Lines for Robust Lentiviral Gene Transfer into Hematopoietic Stem Cells and T-cells. Molecular Therapy 24:1237-1246 (2016). When titer is assessed on a cultured cell line that is continually dividing, no stimulation is required and hence the measured titer is not influenced by surface engineering of the lentiviral particle. Other methods for assessing the efficiency of lentiviral vector systems are provided in Gaererts et al. Comparison of lentiviral vector titration methods. BMC Biotechnol. 6:34 (2006).

It is widely known that lentiviral vector systems have limited efficiency and that attempts to alter the lentiviral vector system often result in decreased efficency. The present inventors have surprisingly discovered that the envelope plasmid of lentiviral vector systems (e.g. a third-generation system) can be modified to encode a plurality of polypeptides in addition to the fusion glycoprotein or functional variant thereof.

In some cases, the vectors and packaging cell lines of the disclosure are capable of generating surface-engineered lentiviral particles at titres of at least about 1×10⁶ IU/mL, at least about 2×10⁶ IU/mL, at least about 3×10⁶ IU/mL, at least about 4×10⁶ IU/mL, at least about 5×10⁶ IU/mL, at least about 6×10⁶ IU/mL, at least about 7×10⁶ IU/mL, at least about 8×10⁶ IU/mL, at least about 9×10⁶ IU/mL, or at least about 1×10⁷ IU/mL. In some cases, the multicistronic vectors of the disclosure are capable of generating surface-engineered lentiviral particles at titres of at least about 1×10⁷ IU/mL, at least about 2×10⁷ IU/mL, at least about 3×10⁷ IU/mL, at least about 4×10⁷ IU/mL, at least about 5×10⁷ IU/mL, at least about 6×10⁷ IU/mL, at least about 7×10⁷ IU/mL, at least about 8×10⁷ IU/mL, at least about 9×10⁷ IU/mL, or at least about 1×10⁸ IU/mL.

### 1.2 T-Cell/NK-Cell Activation Receptors

The disclosure contemplates nucleic acid vectors, lentiviral vectors, and AAV vectors that encode T-cell/NK-cell activation receptors. As used herein, the term “T-cell/NK-cell activation receptors” refers to one or more transmembrane proteins that are configured to be expressed on the cell surface of transduced cells such that the T-cell/NK-cell activation receptor provides a mitogenic signal to the transduced cell. A T-cell/NK-cell activation receptor is used because the target cells are, in most cases, T cells or NK cells. The present methods can be adapted for use with other cell types by use of an activation receptor that retains activity in another cell type. T-cell/NK-cell activation receptors useful here may include a signaling domain that is a cytokine receptor signaling domain, a co-stimulatory receptor signaling domain, a T-cell receptor subunit signaling domain, an NK-cell receptor subunit signaling domain, a growth factor receptor signaling domain, or the like.

### 1.2.1 Non-Limiting Examples of T-cell/NK-cell Activation Receptors

The signaling domain used may be that of a common cytokine receptor gamma chain or that of a common cytokine receptor beta chain. The signaling domain may include an ITAM or a tyrosine capable of binding an SH2-domain when the tyrosine is phosphorylated. In some cases, signaling may be triggered by homo-or heterodimerization of the activation receptor. Phosphorylation of one or more tyrosine residues on an intracellular domain of an activation receptor may, in some cases, lead to dimerization with an SH2 domain and thereby initiate a mitogenic signaling cascade. In some cases a signaling domain of the present disclosure may be capable of being phosphorylated on a tyrosine residue and thereafter binding to an SH2 domain on another molecule.

In some cases, the T-cell/NK-cell activation receptor is a naturally occurring activation receptor. Alternatively, a composite of one or more gene elements taken from different activation receptors known in the art. Examples of engineered T-cell/NK-cell activation receptors useful in the present invention include, without limitation, constitutively active IL2 receptors or IL7 receptors, as described in Hunter et al. Chimeric γc cytokine receptors confer cytokine independent engraftment of human T lymphocytes. Mol Immunol. 2013 November; 56(1-2):1-11.; Shum et al. Constitutive Signaling from an Engineered IL7 Receptor Promotes Durable Tumor Elimination by Tumor-Redirected T Cells. Cancer Discov. 2017 November; 7(11):1238-1247.

### 1.2.2 Small-molecule Controllable T-cell/NK-cell Activation Receptors

It may, in some cases, be advantageous to provide a means to control expansion of transduced cells in the body. In some cases, this could serve as a failsafe to guard again excessive expansion of cells. In other cases, one could control expansion of transduced cells for therapeutic purposes. Thus, the present disclosure provides T-cell/NK-cell activation receptors that are controllable by use of a small molecule. When the lentiviral vector encodes such a controllable T-cell/NK-cell activation receptor, administering the small molecule permits activation of the activation receptor to provide a mitogenic signal to transduced cells, whereas ceasing administration of the small molecule prevents the activation receptor from providing a mitogenic signal to transduced cells. In this way, in vivo TILs generated by administering the lentiviral particles to a subject will expand only while the small molecule is present in the subject. The small molecule can be provided systemically or locally and concurrently with or during the period after administration of the lentiviral particle. Expansion of TILs can be monitored through blood samples, biopsy, or medical imaging, and the small molecule withdrawn if excessive expansion is observed. In some cases, pulsed or intermittent administration of the small molecule may used to optimize the treatment protocol. In some cases, the small molecule will be titrated to tune TIL expansion. In some cases, the small molecule may be withdrawn or administered in response to remission or relapse of the tumor or for other therapeutic reasons.

In some cases, T-cell/NK-cell activation receptors may be configured to be controllable by a small molecule by substitution of the extracellular domains of a dimeric T-cell/NK-cell activation receptor with protein subunits that inducibly dimerize in the presence of a small molecule, or that inducibly dimerize in the absence of a small molecule and return to monomeric state upon removal, degradation, or dilution of the small molecule. It is contemplated that in the case of a heterodimeric T-cell/NK-cell activation receptor, the T-cell/NK-cell activation receptor may be modified so that the extracellular domain of one unit of the T-cell/NK-cell activation receptor comprises one monomer of the protein subunits that inducibly dimerize, and the extracellular domain of the other unit of the T-cell/NK-cell activation receptor comprises the other monomer of the protein subunits that inducibly dimerize. A heterodimeric T-cell/NK-cell activation receptor may be modified in this manner using either a homodimeric protein subunits that inducibly dimerize or heterodimeric protein subunits that inducibly dimerize. In other cases, a homodimeric T-cell/NK-cell activation receptor may be modified in this manner using either homodimeric protein subunits that inducibly dimerize or heterodimeric protein subunits that inducibly dimerize. The small-molecule controllable T-cell/NK-cell activation receptor may be encoded by a single transgene if it is homodimeric or by two transgenes when it is heterodimeric.

Examples of protein subunits that inducibly dimerize include, without limitation, FK506-binding protein (FKBP) and the FKBP12-rapamycin binding (FRB) domain. FK506-binding protein dimerizes in the presence tacrolimus (FK506). FKBP12-rapamycin binding (FRB) domain dimerizes in the presence of rapamycin. Thus, the small molecule according to the present disclosure may be either tacrolimus, rapamycin, or a rapalog (a rapamycin analog). Further examples of small molecules by which small-molecule controllable receptors can be controlled include, without limitation, rapamycin, a rapalog, coumermycin, gibberellin, abscisic acid (ABA), methotrexate, cyclosporin A, FKCSA, trimethoprim (Tmp)-synthetic ligand for FKBP (SLF), or any derivatives thereof. Exemplary pairs of domains that suitable as fusion proteins in the receptors of the present disclosure include, without limitation, a pair selected from FKBP and FRB, FKBP and calcincurin, FKBP and cyclophilin, FKBP and bacterial DHFR, calcincurin and cyclophilin, PYL1 and ABI1, or GIB1 and GAI, or variants thereof.

In some cases, the small-molecule controllable receptor comprises an extracellular domain comprises one or more small-molecule binding domains. Binding for the small-molecule to the extracellular domain(s) causes intramolecular interaction of receptor molecules (e.g. homodimerization or heterodimerization), thus activating a downstream signal from the intracellular domain(s). The intracellular domain(s) of small-molecule controllable receptor may comprise one or more domains selected from domains or fragments of CD28, OX-40, 4-1BB/CD137, CD2, CD7, CD27, CD30, CD40, Programmed Death-1 (PD-1), inducible T cell co-stimulator (ICOS), lymphocyte function-associated antigen-1 (LFA-1, CD1-1a/CD18), CD3 gamma, CD3 delta, CD3 epsilon, CD3 zeta, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, IL-21R alpha, ITGA4, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, CD27, CD28, ICOS, 4-1BB, CD40, RANK/TRANCE-R, OX40, TGFbR1, TGFbRII, myd88, CD40 and any other TNF receptor superfamily members, and combinations thereof.

A “homolog” of a protein of interest, such as FKBP or FRB, includes proteins comprising or consisting of an amino acid sequence that has at least about 70%, 80%, 90%, 95%, 98% or 99% identity with the amino acid sequence of the protein. A homolog may also be a protein that is encoded by a nucleic acid that has at least about 70%, 80%, 90%, 95%, 98% or 99% identity with a nucleotide sequence.

A “functional homolog” of a protein of interest refers to a homolog of the protein having at least one biological activity of the protein. For example, a functional homolog of FKPB refers to a homolog of FKBP that heterodimerizes with FRB in the presence of rapamycin (or related rapalogs) or homodimerizes in the presence of molecules such as AP1903; a functional homolog of FRB refers to a homolog of FRB that heterodimerizes with FKBP in the presence of rapamycin (or related rapalogs).

In some embodiments, the T-cell/NK-cell activation receptor comprises (a) a first chain comprising either or both of: (i) a functional FKPB domain that shares at least 95%, 99%, or 100% sequence identity with SEQ ID NO: 13, and (ii) a functional IL2Rb domain that shares at least 95%, 99%, or 100% sequence identity with SEQ ID NO: 14; and/or (b) a second chain comprising either or both of: (i) a functional FRB domain that shares at least 95%, 99%, or 100% sequence identity with SEQ ID NO: 16, and (ii) a functional IL2Rg domain that shares at least 95%, 99%, or 100% sequence identity with SEQ ID NO: 17.

In some embodiments, the T-cell/NK-cell activation receptor comprises (a) a first chain comprising a functional FKPB domain and a functional IL2Rb domain, wherein the first chain shares at least 95%, 99%, or 100% sequence identity with SEQ ID NO: 12; and/or (b) a second chain comprising a functional FRB domain and a functional IL2Rg domain, wherein the second chain shares at least 95%, 99%, or 100% sequence identity with SEQ ID NO: 15.

### 1.2.3 Chimeric Antigen Receptors

It may, in some cases, be advantageous to provide a means to target transduced cells to particular cells or tissues. In some cases, the lentiviral vector comprises (instead of or in addition to other genes) a polynucleotide encoding a chimeric antigen receptor (CAR). Various CARs known in the art can be employed. Alternatively, T-cell receptor (TCR) fusions can be used. When the vector encodes a CAR, the CAR can comprise one or more domains selected from domain or fragments of CD28, OX-40, 4-1BB/CD137, CD2, CD7, CD27, CD30, CD40, Programmed Death-1 (PD-1), inducible T cell co-stimulator (ICOS), lymphocyte function associated antigen-1 (LFA-1, CD1-1a/CD18), CD3 gamma, CD3 delta, CD3 epsilon, CD3 zeta, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, IL-21R alpha, ITGA4, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, CD27, CD28, ICOS, 4-1BB, CD40, RANK/TRANCE-R, OX40, TGFbR1, TGFbRII, myd88, other TNF receptor superfamily members not already listed, and combinations thereof.

### 1.2.4 Small-Molecule Controllable Chimeric Antigen Receptors

It may, in some cases, be advantageous to provide a means to control targeted of transduced cells. In some cases, this could serve as a failsafe to guard again excessive activity of cells. In other cases, one could control activity of transduced cells for therapeutic purposes. Thus, the present disclosure provides CARs, or similar targeting receptors e.g. TCR fusions, that are controllable by use of a small molecule. When the lentiviral vector encodes such a controllable targeting receptor, administering the small molecule permits activation of the targeting receptor to target transduced cells to target cells, whereas ceasing administration of the small molecule prevents the receptor from targeting transduced cells to target cells. In this way, in vivo TILs generated by administering the lentiviral particles to a subject will activate only while the small molecule is present in the subject. The small molecule can be provided systemically or locally and concurrently with or during the period after administration of the lentiviral particle. Activity of TILs can be monitored through blood samples, biopsy, or medical imaging, and the small molecule withdrawn if excessive activity is observed. In some cases, pulsed or intermittent administration of the small molecule may used to optimize the treatment protocol. In some cases, the small molecule will be titrated to tune TIL activity. In some cases, the small molecule may be withdrawn or administered in response to remission or relapse of the tumor or for other therapeutic reasons.

In some cases, targetting receptors (e.g. CARs) may be configured to be controllable by a small molecule by fusion to protein subunits that inducibly dimerize in the presence of a small molecule, or that inducibly dimerize in the absence of a small molecule and return to monomeric state upon removal, degradation, or dilution of the small molecule. It is contemplated that in the case of a heterodimeric targeting receptors, the T-cell/NK-cell activation receptor may be modified so that the extracellular domain of one unit of the targeting receptor comprises one monomer of the protein subunits that inducibly dimerize, and the extracellular domain of the other unit of the targeting receptor comprises the other monomer of the protein subunits that inducibly dimerize. A heterodimeric targeting receptor may be modified in this manner using either a homodimeric protein subunits that inducibly dimerize or heterodimeric protein subunits that inducibly dimerize. In other cases, a homodimeric targeting receptor may be modified in this manner using either homodimeric protein subunits that inducibly dimerize or heterodimeric protein subunits that inducibly dimerize. The small-molecule controllable targeting receptor may be encoded by a single transgene if it is homodimeric or by two transgenes when it is heterodimeric.

Examples of protein subunits that inducibly dimerize include, without limitation, FK506-binding protein (FKBP) and the FKBP12-rapamycin binding (FRB) domain. FK506-binding protein dimerizes in the presence tacrolimus (FK506). FKBP12-rapamycin binding (FRB) domain dimerizes in the presence of rapamycin. Thus, the small molecule according to the present disclosure may be either tacrolimus, rapamycin, or a rapalog (a rapamycin analog). Further examples of small molecules by which small-molecule controllable receptors can be controlled include, without limitation, rapamycin, a rapalog, coumermycin, gibberellin, abscisic acid (ABA), methotrexate, cyclosporin A, FKCSA, trimethoprim (Tmp)-synthetic ligand for FKBP (SLF), or any derivatives thereof. Exemplary pairs of domains that suitable as fusion proteins in the receptors of the present disclosure include, without limitation, a pair selected from FKBP and FRB, FKBP and calcineurin, FKBP and cyclophilin, FKBP and bacterial DHFR, calcineurin and cyclophilin, PYL1 and ABI1, or GIB1 and GA1, or variants thereof.

In some cases, the small-molecule controllable targeting receptor comprises an extracellular domain comprising one or more small-molecule binding domains. Binding of the small-molecule to the extracellular domain(s) causes intramolecular interaction of receptor molecules (e.g. homodimerization or heterodimerization), thus activating binding to target cells and/or a downstream signal from the intracellular domain(s). The intracellular domain(s) of small-molecule controllable receptor may comprise one or more domains selected from domains or fragments of CD28, OX-40, 4-1BB/CD137, CD2, CD7, CD27, CD30, CD40, Programmed Death-1 (PD-1), inducible T cell co-stimulator (ICOS), lymphocyte function-associated antigen-1 (LFA-1, CD1-1a/CD18), CD3 gamma, CD3 delta, CD3 epsilon, CD3 zeta, CD247, CD276 (B7-H3), LIGHT, (TNFSF14), NKG2C, Ig alpha (CD79a), DAP-10, Fc gamma receptor, MHC class 1 molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, IL-21R alpha, ITGA4, VLA1, CD49a, ITGA4, IA4, CD49D, ITGA6, VLA-6, CD49f, ITGAD, CD1 1d, ITGAE, CD103, ITGAL, CD1 1a, LFA-1, ITGAM, CD1 1b, ITGAX, CD1 1c, ITGB1, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, CD27, CD28, ICOS, 4-1BB, CD40, RANK/TRANCE-R, OX40, TGFbR1, TGFbRII, myd88, CD40 and any other TNF receptor superfamily members, and combinations thereof. Further exemplary controllable CARs are provided by U.S. Pat. No. 10,196,444, incorporated herein by reference.

In some embodiments, the two protein subunits that inducibly dimerize are each fused to a transmembrane protein comprising signaling domain(s) and to a binding agent (e.g. single-chain variable fragment, scFv), respectively. The binding agent is capable of specifically binding to a target of interest. Small-molecule induced dimerization reconstitutes a dimeric unit spanning from the binding agent through the dimerized protein subunits to the intracellular signaling domain(s) via the transmembrane region of the transmembrane protein. In some embodiments, two transmembrane proteins are employed and inducible dimerize to one another and to the binding-agent fusion protein in response to one or two small molecules. In some embodiments, the small-molecule controllable CAR comprises two or more binding agent fusion proteins that each individually dimerize to the transmembrane protein comprising signaling domain(s).

### 1.4 Promoters and Gene Control Elements

The present disclosure further contemplates lentiviral vectors comprising promoters and/or enhancers specific to T-cells, NK cells, or T-cells and NK cells. The present disclosure provides nucleic acid sequences of T-cell and/or NK-cell specific promoters (SEQ ID NOs: 1-4). These can be operatively linked to the T-cell/NK-cell activation receptor generally by inserting the promoter sequence 5′ to the gene encoded by the lentiviral vector. The promoter use can be identical to a sequence set forth in SEQ ID NOs: 1-4 or 80%, 85%, 90%, 95%, or 99% identical to a sequence set forth in SEQ ID NOs: 1-4, so long as the promoter retains promoter activity in T and/or NK cells.

In some cases, other promoters can used to control expression of the T-cell/NK-cell activation receptor. Examples of promoter useful in the present disclosure include, without limitation, an MND promoter, a T-cell specific promoter, a CD4 T-cell specific promoter, a CD8 T-cell specific promoter, an NK-cell specific promoter, a T-cell and NK-cell specific promoter, a CD4 T-cell and NK-cell specific promoter, and a CD8 T-cell and NK-cell specific promoter.

In some cases, a “strong” promoter is used. It will be understood that the strength of a promoter is determined in part by the attributes of the cell in which it operates. In some cases, the strong promoter of the present disclosure results in high-level expression of gene elements to which it is operatively linked in a target cell, such as a TIL. Strong promoters include, without limitation, cytomegalovirus (CMV) and murine stem cell virus (MSCV), phosphoglycerate kinase (PGK), a promoter sequence comprised of the CMV enhancer and portions of the chicken beta-actin promoter and the rabbit beta-globin gene (CAG), promoter sequence comprised of portions of the SV40 promoter and CD43 promoter (SV40/CD43), and a synthetic promoter that contains the U3 region of a modified MoMuLV LTR with myeloproliferative sarcoma virus enhancer (MND). Exemplary strong promoters useful in the compositions and method of the present disclosure are provided by Jones et al. Lentiviral vector design for optimal T cell receptor gene expression in the transduction of peripheral blood lymphocytes and tumor-infiltrating lymphocytes. Hum Gene Ther. 2009 June; 20(6):630-40.

In some cases, the strong promoter may be a synthetic strong promoter. Exemplary synthetic strong promoters are provided by Schlabach et al. (2010) Proc Natl Acad Sci USA. 10:2538-2543. In some cases, other promoters are used. In some cases, any promoter active in the packaging cell line is used. In some cases, an inducible promoter is used, e.g. a drug-inducible promoter.

In some cases, vectors of the present disclosure may comprise the Woodchuck Hepatitis Virus Posttranscriptional Regulatory Element (wPRE) or a nucleic acid sequence substantially identical to wPRE. See U.S. Pat. No. 6,136,597; Lee et al. (2005) Exp Physiol. 90:33-7. Variants of the wPRE element with reduced size are known in the art. wPRE-O refers to a variant of wPRE with the intermediate size.

In some cases, lentiviral vectors of the present disclosure may comprise a polynucleotide sequence encoding the 2A peptide. The term “2A peptide” refers to a self-cleaving peptide configured to generate two or more proteins from a single open reading frame. 2A peptides are 18-22 residue-long viral oligopeptides that mediate “cleavage” of polypeptides during translation in eukaryotic cells. “2A peptide” may refer to peptides with various amino acid sequences. In the present disclosure it will be understood that where a lentiviral vector comprises two or more 2A peptides, the 2A peptides may be identical to one another or different. Detailed methodology for design and use of 2A peptides is provided by Szymczak-Workman et al. (2012) Cold Spring Harb. Protoc. 2012:199-204. In the literature, 2A peptides are often refered to as self-cleaving peptides, but mechanistic studies have shown that the “self-cleavage” observed is actually a consequence of the ribosome's skipping the formation of the glycyl-prolyl peptide bond at the C terminus of the 2A peptide. Donnelly et al. (2001) J Gen Virol. 82:1027-41.

In some cases, lentiviral vectors of the present disclosure may comprise the Woodchuck Hepatitis Virus Posttranscriptional Regulatory Element (wPRE) or a nucleic acid sequence substantially identical to wPRE. See Lee et al. Optimizing regulatable gene expression using adenoviral vectors. Exp Physiol. 90(1):33-7 (2005). In the viral vectors of the present disclosure, the wPRE sequence increases expression of genes delivered by said viral vectors.

### 1.5 Fusion Glycoproteins

Various fusion glycoproteins can be used to pseudotype lentiviral vecotrs. While the most commonly used example is the envelope glycoprotein from vesicular stomatitis virus (VSVG), many other viral proteins have also been used for pseudotyping of lentiviral vectors. Sec Joglekar et al. (2017) Human Gene Therapy Methods 28:291-301. The present disclosure contemplates substitution of various fusion glycoproteins. Notably, some fusion glycoproteins result in higher vector efficiency.

In some embodiments, pseudotyping a fusion glycoprotein or functional variant thereof facilitates targeted transduction of specific cell types, including, but not limited to, T cells or NK-cells. In some embodiments, the fusion glycoprotein or functional variant thereof is/are full-length polypeptide(s), functional fragment(s), homolog(s), or functional variant(s) of Human immunodeficiency virus (HIV) gp160, Murine leukemia virus (MLV) gp70, Gibbon ape leukemia virus (GALV) gp70, Feline leukemia virus (RD114) gp70, Amphotropic retrovirus (Ampho) gp70, 10A1 MLV (10A1) gp70, Ecotropic retrovirus (Eco) gp70, Baboon ape leukemia virus (BaEV) gp70, Measles virus (MV) H and F, Nipah virus (NiV) H and F, Rabies virus (RabV) G, Mokola virus (MOKV) G, Ebola Zaire virus (EboZ) G, Lymphocytic choriomeningitis virus (LCMV) GP1 and GP2, Baculovirus GP64, Chikungunya virus (CHIKV) E1 and E2, Ross River virus (RRV) E1 and E2, Semliki Forest virus (SFV) E1 and E2, Sindbis virus (SV) E1 and E2, Venezualan equine encephalitis virus (VEEV) E1 and E2, Western equine encephalitis virus (WEEV) E1 and E2, Influenza A, B, C, or D HA, Fowl Plague Virus (FPV) HA, Vesicular stomatitis virus VSV-G, or Chandipura virus and Piry virus CNV-G and PRV-G. In some cases, the fusion glycoprotein or functional variant thereof is a full-length polypeptide, functional fragment, homolog, or functional variant of the G protein of Vesicular Stomatitis Alagoas Virus (VSAV), Carajas Vesiculovirus (CJSV), Chandipura Vesiculovirus (CHPV), Cocal Vesiculovirus (COCV), Vesicular Stomatitis Indiana Virus (VSIV), Isfahan Vesiculovirus (ISFV), Maraba Vesiculovirus (MARAV), Vesicular Stomatitis New Jersey virus (VSNJV), Bas-Congo Virus (BASV). In some embodiments, the fusion glycoprotein or functional variant thereof is the Cocal virus G protein.

### 1.6 Checkpoint-Inhibiting Ligands

In some cases, the lentiviral vectors of the present disclosure further comprise a nucleic acid sequence encoding a checkpoint-inhibiting ligand. Optionally, the checkpoint-inhibiting ligand is capable of blocking the PD-1/PD-L1 checkpoint. Optionally, the checkpoint-inhibiting ligand is capable of blocking the Tim-3 checkpoint.

Checkpoint inhibitor therapy is a form of cancer treatment that uses agents to stimulate or inhibit immune checkpoints and thereby modulate the immune response. Tumors may use checkpoints to protect themselves from the immune system of the subject or from therapeutic agents used in cancer immunotherapy. The present disclosure provides a lentiviral vector comprising a nucleic acid sequence encoding a checkpoint-inhibiting ligand, wherein lentiviral particles produced from the lentiviral vector display the checkpoint-inhibiting ligand on their surface, and therefore administration of the lentiviral particle results in delivery of the checkpoint-inhibiting ligand to the subject at the site of therapeutic use. The present disclosure further provides lentiviral vectors comprising a nucleic acid sequence encoding a checkpoint-inhibiting ligand, wherein administration of lentiviral particles produced from the lentiviral vector delivers the polynucleotide sequence to target cells, which then express the checkpoint-inhibiting ligand at the site of therapeutic use.

Examples of checkpoint-inhibitor ligands provided by the present disclosure include, without limitation, anti-CTLA-4 antibody, anti-PD-1 antibodies, and anti-PD-L1 antibodies or any non-antibody ligands (e.g. nanobodies, DARPins) that interact with CTLA4, PD-1, or PD-L1 respectively. In some cases, the checkpoint-inhibiting ligand is capable of blocking the PD-1/PD-L1 checkpoint and/or the Tim-3 checkpoint and/or the CTLA-4 checkpoint. Use of checkpoint inhibition is reviewed in, e.g., Anderson et al. Tim-3: an emerging target in the cancer immunotherapy landscape. Cancer Immunol Res. 2014 May; 2(5):393-8.

### 1.5 Resistance to Immunosuppressive Drugs

In some cases, the lentiviral vectors of the present disclosure further comprises a nucleic acid sequence (e.g., on the transfer plasmid) that provides resistance to a immunosuppressive drug. A nucleic acid sequence that provides resistance to a immunosuppressive drug will, in some cases, facilitate selective expansion of target cells when the immunosuppressive drug is administered to a patient during any of the methods for treating a subject or any of the methods for expanding T-cells capable of recognizing and killing tumor cells in a subject in need thereof provided by the present disclosure. In some cases, the immunosuppressive drug is methotrexate, rapamycin, a rapalog, tacrolimus, cyclosporine, or any combination thereof. The immunosuppressive drug may be the same as the small molecule or different, that is the lentiviral vector may be designed so that the small-molecule controllable T-cell/NK-cell activation receptors is induced by an immunosuppressive drug such that whenever the immunosuppressive drug is administered to the subject, expansion of transduced cells is triggered. Alternatively, it may be advantageous to design the lentiviral vector to permit control of transduced-cell expansion independent of immunosuppression.

In some cases, the lentiviral vector facilitates selective expansion of target cellsby conferring resistance to an immunosuppressive drug to transduced cells, facilitates selective expansion of target cells. The present disclosure provides lentiviral vectors that comprise any of the nucleic sequences that confer resistance to an immunosuppressive drug known in the art. Examples of immunosuppressive drugs include, without limitation, rapamycin or a derivative thereof, a rapalog or a derivative thereof, tacrolimus or a derivative thereof, cyclosporine or a derivative thereof, methotrexate or derivatives thereof, and mycophenolate mofetil (MMF) or derivatives thereof. Various resistance genes are known in the art. Resistance to rapamycin may be conferred by a polynucleotide sequence encoding the protein domain FRb, found in the mTOR domain and known to be the target of the FKBP-rapamycin complex. Resistance to tacrolimus may be conferred by a polynucleotide sequence encoding the calcineurin mutant CNa22 or calcineurin mutant CNb30. Resistance to cyclosporine may be conferred by a polynucleotide sequence encoding the calcineurin mutant CNa12 or calcineurin mutant CNb30. These calcineurin mutants are described in Brewin et al. (2009) Blood 114:4792-803. Resistance to methotrexate can be provided by various mutant forms of di-hydrofolate reducatse (DHFR), see Volpato et al. (2011) J Mol Recognition 24:188-198, and reistance to MMF can be provided by various mutant forms of inosine monophosphate dehydrogenase (IMPDH), see Yam et al. (2006) Mol Ther 14:236-244.

Immunosuppressive drugs are commonly used prior to, during, and/or after ACT. In some cases, use of an immunosuppressive drug may improve treatment outcomes. In some cases, use of an immunosuppressive drug may diminish side effects of treatment, such as, without limitation, acute graft-versus-host disease, chronic graft-versus-host disease, and post-transplant lymphoproliferative disease. The present disclosure contemplates use of immunosuppressive drugs with any of the methods of treating or preventing a disease or condition of the present disclosure, including, without limitation, methods of the present disclosure in which the lentiviral vector confers resistance to an immunosuppressive drug to transduced cells.

### 2.1 Packaging Cell Lines

In another aspect, the present disclosure provides packaging cell lines for generating lentiviral particles capable of activating and efficiently transducing T cells, comprising cultured cells capable of packaging a lentivirus vector, wherein the cultured cells are genetically engineered to express a T-cell activation or co-stimulation molecule, or are induced to transiently express a T-cell activation or co-stimulation molecule via transient transfection. The packaging cell lines of the present disclosure may be used with any lentiviral vector including but not limited to those previously described. In some cases, it will be advantageous to use the packaging cell line with a lentiviral vector comprising a nucleic acid sequence encoding a T-cell/NK-cell activation receptor. In some cases, it will be advantageous for that T-cell/NK-cell activation receptor to be capable of being activated by a small molecule. But the packaging cell lines disclosed can be used with other lentiviral vectors as well.

In some cases, the packaging cell line is a HEK-293T cell line. Similar results can be achieved with other cell lines, including, without limitation, HEK-293T cell lines engineered to be deficient in B2M or other immunologically active surface proteins. Other cell lines that are transfectable in vitro and capable of high titer lentiviral vector production cand be used—e.g., cell lines that comprise the gene sequence for polyoma virus large T antigen operatively linked to a promoter.

The packaging cell line may be, in some cases, genetically modified to lack MHC class I expression, MHC class II expression, or expression of inhibitory checkpoint ligands, such as PD-L1 (a PD-1 ligand), or ligands for TIM3. As expression of inhibitory ligands by the packaging cell line could limit T-cell activation by the lentiviral particles, these genetic modification serve in some cases to eliminate such inhibitory signals, further promoting T-cell activation and transduction by the lentiviral particles.

In some cases, a packaging cell line is genetically engineered to comprise one or more genes useful in packing lentiviral vectors into lentiviral particles. In some cases, a packaging cell line may comprise polynucleotide sequences encoding the genes gag-pol, env, and rev. In a typical lentiviral vector of the present invention, at least part of one or more of the gag-pol and env protein coding regions may be removed from the lentiviral vector and provided by the packing cell line. Lentiviral vectors may be packaged according to the methods provided in Dull et al. (1998) J Virol 72:8463-71, which is incorporated herein in its entirety. Exemplary packaging cell lines are provide in Retroviruses. Cold Spring Harbour Laboratory (Coffin et al., eds) (1997).

The present disclosure further provides for genetically engineering the packaging cell line to improve immunological attributes of the lentiviral vectors and particles of the present disclosure in other ways, including, without limitation, adding genes, deleting genes, and introducing point mutations into genes.

### 2.2 T-cell Activation or Co-Simulation Molecules

Conventionally, lentiviral transduction in vitro requires additional of an exogenous activating agent, such as a “stimbead,” for example Dynabeads™ Human T-Activator CD3/CD28. Lentiviral particles made using the packaging cell lines of the present disclosure incorporate one or more copies of the T-cell activation or co-stimulation molecule that is expressed by the packaging cell line into the lentiviral particle; and the incorporation of T-cell activation or co-stimulation molecule(s) in the lentiviral particle renders the lentiviral particle capable of activating and efficiently transducing T cells in the absence of an exogenous activating agent, i.e. without a stimbead or equivalent agent. This permits the lentiviral particles made from these packaging cell lines to be used in vivo in cases in which exogenous delivery of an activating agent may be impractical.

In some cases, the T-cell activation or co-stimulation molecule may be selected from the group consisting of an anti-CD3 antibody, CD28 ligand (CD28L), and 41bb ligand (41BBL or CD137L). Various T-cell activation or co-stimulation molecules are known in the art and include, without limitation, agents that specifically bind any of the T-cell expressed proteins CD3, CD28, CD134 also known as OX40, or 41bb also known as 4-1BB or CD137 or TNFRSF9. For example, an agent that specifically binds CD3 may be an anti-CD3 antibody (e.g., OKT3, CRIS-7 or I2C) or an antigen-binding fragment of an anti-CD3 antibody. In some aspects, an agent that specifically binds CD3 is a single chain Fv fragment (scFv) of an anti-CD3 antibody. In some cases, the present disclosure contemplates that the T-cell activation or co-stimulation molecule is selected from the group consisting of an anti-CD3 antibody, CD28 ligand (CD28L), and 41bb ligand (41BBL or CD137L). CD86 also known as B7-2 is a ligand for both CD28 and CTLA-4. In case cases, a CD28L may be CD86. CD80 is an additional ligand for CD28. In some cases, the ligand for CD28 is CD80. In some cases, the ligand for CD28 is an anti-CD28 antibody or an anti-CD28 scFv fused to a transmembrane domain for display on the surface of the lentiviral particle. Lentiviral particles comprising one or more a T-cell activation or co-stimulation molecules may be made by methods provided by WO2016/139463.

### 3 Lentiviral Particles

In another aspect the present disclosure further provides lentiviral particles comprising any of the lentiviral vectors of the present disclosure. The lentiviral particles of the present disclosure can be made with the packaging cell lines of the present disclosure, or with another packaging cell line, or by co-transfection of cultured cells, e.g. HEK-293T cells, with the lentiviral vector and helper plasmids. The lentiviral particles of the present disclosure can be prepared by, for example, transducing any of the lentiviral vectors of the present disclosure into cultured cells genetically engineered to express a T-cell activation or co-stimulation molecule. In some cases, due to the lentiviral vector, to the packaging cell line chosen, or to co-transfected helper plasmids, the lentiviral particle will comprise a T-cell activation or co-stimulation molecule, which molecule could be, without limitation, an anti-CD3 antibody, an CD28 ligand, or 41bb ligand. The cultured cells genetically engineered to express a T-cell activation or co-stimulation molecule could be HEK-293T cells. In some cases, the cultured cells are genetically modified to lack MHC class I expression, MHC class II expression, or expression of inhibitory checkpoint ligands, such as PD-L1 (a PD-1 ligand), or ligands for TIM3.

### 4 Methods of Use

In another aspect, the disclosure provides methods for treating a subject suffering from cancer, comprising administering any lentiviral particle of the present disclosure to the subject, and administering the small molecule to the subject, wherein the cancer is treated in the subject.

In some embodiments, the disclosure provides a surface-engineered lentiviral particle for use in therapy. In other embodiments, the disclosure provides a surface-engineered lentiviral particle for use in a method of treating a cancer. In further embodiments, the disclosure provides a surface-engineered lentiviral particle for use in the manufacture of a medicament for treating cancer.

In some cases, the cancer is a solid tumor, such as a melanoma, non-small cell lung cancer, or breast cancer. The methods of the present disclosure may include treating any cancer, including, without limitation, acute granulocytic leukemia, acute lymphocytic leukemia, acute myelogenous leukemia, adenocarcinoma, adenosarcoma, adrenal cancer, adrenocortical carcinoma, anal cancer, anaplastic astrocytoma, angiosarcoma, appendix cancer, astrocytoma, basal cell carcinoma, B-cell lymphoma, bile duct cancer, bladder cancer, bone cancer, bone marrow cancer, bowel cancer, brain cancer, brain stem glioma, brain tumor, breast cancer, carcinoid tumors, cervical cancer, cholangiocarcinoma, chondrosarcoma, chronic lymphocytic leukemia, chronic myelogenous leukemia, colon cancer, colorectal cancer, craniopharyngioma, cutaneous lymphoma, cutaneous melanoma, diffuse astrocytoma, ductal carcinoma in situ, endometrial cancer, ependymoma, epithelioid sarcoma, esophageal cancer, Ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors, general, germ cell tumor, gestational trophoblastic disease, glioblastoma multiforme, glioma, hairy cell leukemia, head and neck cancer, hemangioendothelioma, Hodgkin lymphoma, Hodgkin lymphoma, Hodgkin's disease, hypopharyngeal cancer, infiltrating ductal carcinoma, infiltrating lobular carcinoma, inflammatory breast cancer, intestinal cancer, intrahepatic bile duct cancer, invasive/infiltrating breast cancer, islet cell cancer, jaw cancer, kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma, metastatic breast cancer, metastatic melanoma, metastatic squamous neck cancer, mixed gliomas, mouth cancer, mucinous carcinoma, mucosal melanoma, multiple myeloma, mycosis fungoides, myelodysplastic syndrome, nasal cavity cancer, nasopharyngeal cancer, neck cancer, neuroblastoma, neuroendocrine tumors, non-Hodgkin lymphoma, non-Hodgkin's lymphoma, non-small cell lung cancer, oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer, ovarian germ cell tumor, ovarian primary peritoneal carcinoma, ovarian sex cord stromal tumor, paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pincoblastoma, pituitary tumors, primary central nervous system, prostate cancer, rectal cancer, renal cell carcinoma, renal pelvis cancer, rhabdomyosarcoma, salivary gland cancer, sarcoma, sarcoma, bone, sarcoma, soft tissue, sarcoma, uterine, sinus cancer, skin cancer, small cell lung cancer, small intestine cancer, soft tissue sarcoma, spinal cancer, spinal column cancer, spinal cord cancer, spinal tumor, squamous cell carcinoma, stomach cancer, synovial sarcoma, t-cell lymphoma, testicular cancer, throat cancer, thymoma/thymic carcinoma, thyroid cancer, tongue cancer, tonsil cancer, transitional cell cancer, transitional cell cancer, transitional cell cancer, triple-negative breast cancer, tubal cancer, tubular carcinoma, undiagnosed cancer, ureteral cancer, ureteral cancer, urethral cancer, uterine adenocarcinoma, uterine cancer, uterine sarcoma, vaginal cancer, vulvar cancer.

In another aspect, the disclosure provides a method for expanding T-cells capable of recognizing and killing tumor cells in a subject in need thereof, including the steps of administering a lentiviral particle of the disclosure to the subject, such that T-cells capable of recognizing and killing tumor cells in the subject are transduced by the lentiviral particle and expanded. In some embodiments, the lentiviral particle is administered by intravenous injection or by intratumoral injection.

In some cases, the lentiviral particle comprises a targeting agent or the nucleic acid vector encodes a targeting agent. Exemplary targeting agents include antibodies and chimeric antigen receptors (“CAR”). The term “antibody” refers to an intact antigen-binding immunoglobulin of any kind, or a fragment thereof that itself specifically binds to the antibody's target antigen, and includes, for example, chimeric, humanized, fully human, and bispecific antibodies. The CAR used in the present disclosure in some cases comprises a binding domain which is specific for B-cells, e.g., specific for a CD-marker that can be found on B-cell lymphoma such as CD19, CD22, CD20 or CD79a, CD19 being preferred. T-cells that have been genetically engineered to express a CAR (e.g., a T-cell CAR) are exemplified in WO2007/131092. The targeting agent serves, in some cases, to direct cell-mediated immunity towards other particular cell types, such as tumor cells.

In another aspect, the disclosure provides methods for expanding T-cells capable of recognizing and killing tumor cells in a subject in need thereof, comprising administering any lentiviral particle of the present disclosure to the subject, and administering the small molecule to the subject, wherein T-cells capable of recognizing and killing tumor cells in the subject are expanded.

In certain embodiments, a subject treated by the methods described herein may be a mammal. In some cases, a subject is a human, a non-human primate, a pig, a horse, a cow, a dog, a cat, a rabbit, a mouse or a rat. A subject may be a human female or a human male.

Combination therapies are also contemplated by the invention. Combination as used herein includes simultaneous treatment or sequential treatment. Combinations of methods of the invention with standard medical treatments (e.g., corticosteroids) are specifically contemplated, as are combinations with novel therapies. In some cases, a subject may be treated with a steroid (e.g. prednisone, prednisolone, deflazacort) to prevent or to reduce an immune response to administration of a lentiviral particle described herein. In certain cases, a subject may receive apheresis or another immune modulator if the subject expresses antibodies to the lentiviral particle described herein. In some cases, such immune modulators may be unnecessary, particularly when an immunosuppressive agent (e.g. tacrolimus or sirolimus) is administered. In some cases, rituxan is administered simultaneous to or sequential with treatment with a lentiviral particles. Rituxan may in some cases serve to block immune responses against the lentiviral particles.

The lentiviral particles, small molecules, and immunosuppressive drugs of the present disclosure may be administered by any route, including oral, nasal, intravenous, intra-arterial, intramuscularly, or intraperitoneal routes. In some cases, the lentiviral particle is administered by intravenous injection or by intratumoral injection. In some cases, the small molecule is administered by intravenous injection or administered orally. In some cases, the small molecule is administered at concentrations sufficient to activate the T-cell/NK-cell activation receptor. In some cases, the small molecule is rapamycin, optionally administered at concentrations sufficient to maintain serum concentrations of rapamycin greater than 0.1 nM, 1 nM, or 10 nM. In some cases, the small molecule is a rapalog, optionally administered at concentrations sufficient to maintain serum concentrations of the rapalog greater than 0.1 nM, 1 nM, or 10 nM. In some cases, the small molecule is capable of causing dimerization of the T-cell/NK-cell activation receptor resulting in a cell activation signal.

In some cases, the small molecule is administered simultaneously with the lentiviral particle; or the small molecule is administered about 30 minutes, about 1 hour, about 2 hours, about 4 hours, about 5 hours or about 10 hours before or after the lentiviral particle is administered. In some cases, the method further comprises administering to the subject an immunosuppressive agent. In some cases, the immunosuppressive agent is tacrolimus, optionally administered at concentrations sufficient to maintain serum concentrations of tacrolimus greater than 0.1 nM, 1 nM, or 10 nM. In some cases, the immunosuppressive agent is cyclosporine, optionally administered at concentrations sufficient to maintain serum concentrations of cyclosporine greater than 0.1 nM, 1 nM, or 10 nM. In some cases, the immunosuppressive agent is an immunosuppressive drug. In some cases, the immunosuppressive agent is an immunosuppressive drug and the lentiviral vector comprises a nucleic acid sequence encoding a protein that provides resistance to said immunosuppressive drug.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required. The injectable solutions may be prepared aseptically or filter-sterilized.

### 5 Definitions

Unless defined otherwise, all technical and scientific terms used herein have the same meaning as commonly understood by those of ordinary skill in the art to which the invention belongs. For the purposes of the present invention, the following terms are defined below.

As used herein, “293T control particles” or “Mock (293T vector)” refer to lentiviral particles generated by transduction of 293T cells with a lentiviral vector. As used herein, “stimbeads” refers to a bead-based reagent used to stimulate T-cells during transduction. The label “+Control (vector +stimbeads)” refers transduction with 293T control particles with stimbeads.

As used herein, the term “HATSE cells” or “HATSE cell line” or “HATSE-293” refers a packaging cell line created by transducing 293T cells with lentiviral vector(s) encoding CD86 and CD137L and subjected to fluorescence-activated cell sorting (FACS) for cells that highly expressed both CD86 and CD137L one or more times. The label “(1× sorted) HATSE cell vector” refers to lentiviral particles generated by transduction of HATSE cells with a lentiviral vector after the HATSE cells are FACS-sorted for CD86⁺/CD173L⁺ double-positive cells one time. The label “(2× sorted) HATSE cell vector refers to lentiviral particles generated by transduction of HATSE cells with a lentiviral vector after the HATSE cells are FACS-sorted for CD86⁺/CD173^{L+}double-positive cells one time.

The articles “a,” “an,” and “the” are used herein to refer to one or to more than one (i.e. to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, +4%, ±3%, ±2%, or ±1% about a reference quantity, level, value, number, frequency, percentage, dimension, size, amount, weight or length.

Any concentration range, percentage range, ratio range, or integer range is to be understood to include the value of any integer within the recited range and, when appropriate, fractions thereof (such as one tenth and one hundredth of an integer), unless otherwise indicated. The term “about”, when immediately preceding a number or numeral, means that the number or numeral ranges plus or minus 10%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. In particular embodiments, the terms “include,” “has,” “contains,” and “comprise” are used synonymously.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure, or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “isolated” means material that is substantially or essentially free from components that normally accompany it in its native state. In particular embodiments, the term “obtained” or “derived” is used synonymously with isolated.

A “subject,” “patient” or “individual” as used herein, includes any animal that exhibits pain that can be treated with the vectors, compositions, and methods contemplated herein. Suitable subjects (e.g., patients) include laboratory animals (such as mouse, rat, rabbit, or guinea pig), farm animals, and domestic animals or pets (such as a cat or dog). Non-human primates and, preferably, human patients, are included.

As used herein “treatment” or “treating,” includes any beneficial or desirable effect associated with treatment. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of the disorder. As used herein, “prevention” and similar words also includes reducing the intensity, effect, symptoms and/or burden of the disease or disorder prior to onset or recurrence.

As used herein, “therapeutically effective amount” or “amount effective” or “effective amount” of a virus or lentiviral particle refers to the amount of the virus or lentiviral particle required to achieve a beneficial or desired prophylactic or therapeutic result, including clinical results.

A “prophylactically effective amount” refers to an amount of a virus or lentiviral particle effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a virus or lentiviral particle may vary according to factors such as the disease state, age, sex, and weight of the individual, and the ability of the stem and progenitor cells to elicit a desired response in the individual. A therapeutically effective amount is also one in which any toxic or detrimental effects of the virus are outweighed by the therapeutically beneficial effects. The term “therapeutically effective amount” includes an amount that is effective to “treat” a subject (e.g., a patient).

An “increased” or “enhanced” amount of a physiological response, e.g., electrophysiological activity or cellular activity, is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated subject.

A “decrease” or “reduced” amount of a physiological response, e.g., electrophysiological activity or cellular activity, is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the level of activity in an untreated subject.

By “maintain,” or “preserve,” or “maintenance,” or “no change,” or “no substantial change,” or “no substantial decrease” refers generally to a physiological response that is comparable to a response caused by either vehicle, or a control molecule/composition. A comparable response is one that is not significantly different or measurable different from the reference response.

“Receptor-ligand binding,” “ligand binding,” and “binding” are used interchangeably herein to mean physical interaction between a receptor and a ligand or a synthetic ligand. Ligand binding can be measured by a variety of methods known in the art (e.g., detection of association with a radioactively labeled ligand).

As used herein, the terms “specific binding affinity” or “specifically binds” or “specifically bound” or “specific binding” are used interchangeably throughout the specification and claims and refer to that binding which occurs between a paired species of molecules, e.g., receptor and ligand. When the interaction of the two species produces a non-covalently bound complex, the binding which occurs is typically electrostatic, hydrogen-bonding, or the result of lipophilic interactions. In various embodiments, the specific binding between one or more species is direct. In one embodiment, the affinity of specific binding is about 2 times greater than background binding (non-specific binding), about 5 times greater than background binding, about 10 times greater than background binding, about 20 times greater than background binding, about 50 times greater than background binding, about 100 times greater than background binding, or about 1000 times greater than background binding or more.

In general, “sequence identity” or “sequence homology” refers to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin And Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (generally nucleotides or amino acids), divided by the total number of symbols in the shorter of the two sequences. The program may be used to determine percent identity over the entire length of the proteins being compared. Default parameters are provided to optimize searches with short query sequences in, for example, with the blastp program. The program also allows use of an SEG filter to mask-off segments of the query sequences as determined by the SEG program of Wootton and Federhen, Computers and Chemistry 17:149-163 (1993). Ranges of desired degrees of sequence identity are approximately 80% to 100% and integer values therebetween. Typically, the percent identities between a disclosed sequence and a claimed sequence are at least 80%, at least 85%, at least 90%, at least 95%, or at least 98%.

The term “exogenous” is used herein to refer to any molecule, including nucleic acids, protein or peptides, small molecular compounds, and the like that originate from outside the organism. In contrast, the term “endogenous” refers to any molecule that originates from inside the organism (i.e., naturally produced by the organism).

The term “MOI” is used herein to refer to multiplicity of infection, which is the ratio of agents (e.g. viral particles) to infection targets (e.g. cells).

All publications and patents mentioned herein are hereby incorporated by reference in their entirety as if each individual publication or patent was specifically and individually indicated to be incorporated by reference. In case of conflict, the present application, including any definitions herein, will control. However, mention of any reference, article, publication, patent, patent publication, and patent application cited herein is not, and should not be taken as an acknowledgment, or any form of suggestion, that they constitute valid prior art or form part of the common general knowledge in any country in the world.

The section headings used herein are for organizational purposes only and are not to be construed as limiting the subject matter described.

The invention is further described in the following Examples, which do not limit the scope of the invention described in the claims.

### EXAMPLES

### Example 1: HATSE-293 Packaging Cell Line

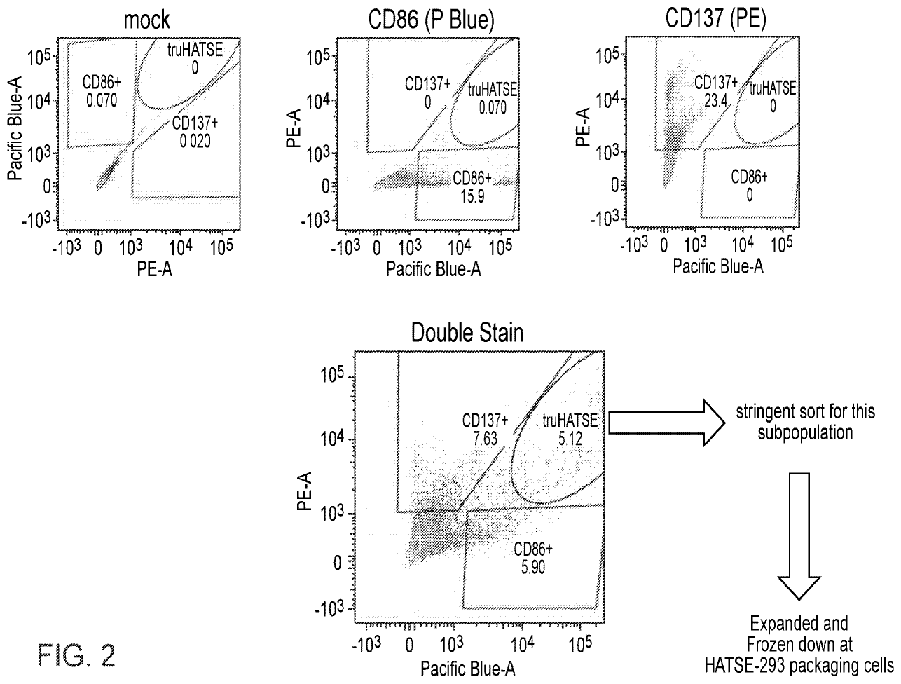

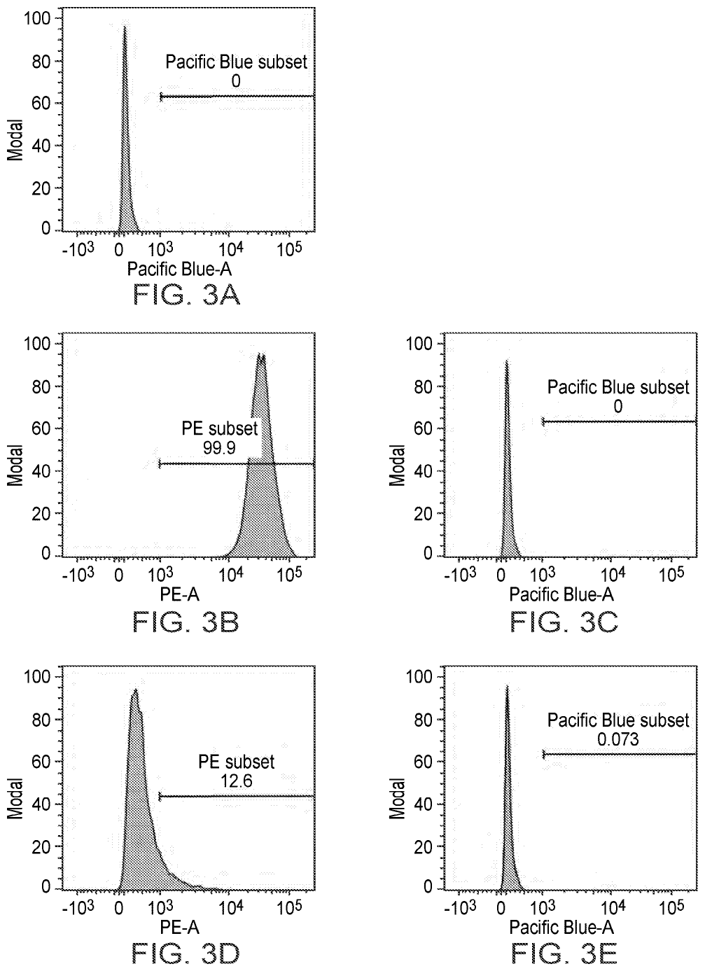

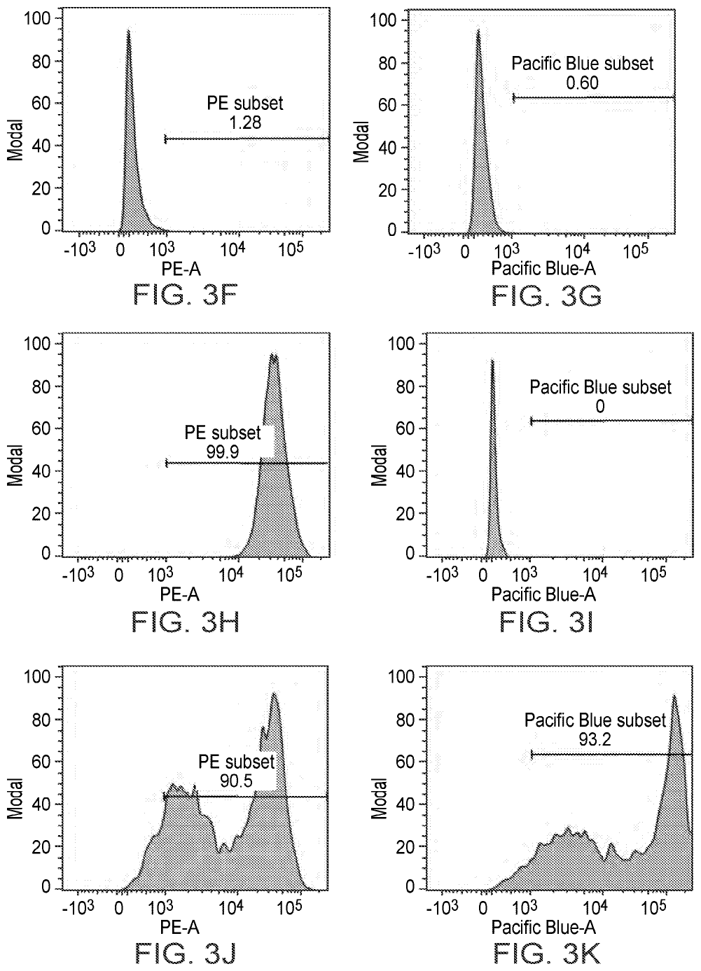

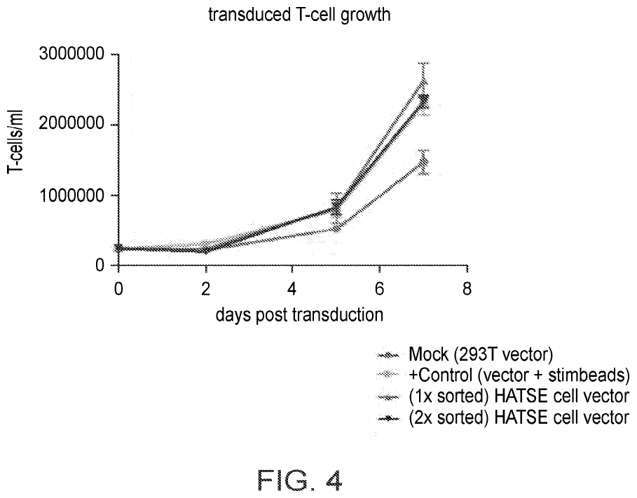

A packaging cell line for generating lentiviral particles capable of activating and efficiently transducing T cells, termed HATSE-293, was generated as follows. A lentiviral vector was constructed comprising the MND promoter and a 2A peptide-linked multicistronic open reading frame encoding an anti-CD3 single chain Fv fragment (scFv) of the monoclonal antibody OKT3; CD86; and CD137L (anti-CD3scFV-2A-CD86-2A-CD137L). The lentiviral vector was transduced into HEK-293T cells grown in cell culture, resulting in stable integration of the MND promoter and multicistronic open reading frame into the genome of host cells. The transduced HEK-293T cells were subjected to fluorescence-activated cell sorting (FACS) for cells that highly expressed both CD86 and CD137L (FIG. 2). Due to the structure of the multicistronic open reading frame, CD86⁺/CD137L⁺ cells also necessarily express the anti-CD3 scFv⁺. The CD86⁺/CD137L⁺ cell population was expanded in culture to produce the HATSE-293 packing cell line and aliquots were frozen for long-term storage and use. The expression of CD86 and CD137 were confirmed by flow cytometry (FIG. 3).

HATSE-293 cells were co-transfected with the pMND-GFP lentiviral vector and four lentiviral packaging plasmids (pMD2.G, pRSV-rev, pMDLG-pRRE, and pRRL-GOI), and viral particles were harvested from the cell supernatant to produce lentiviral particles (termed HATSE particles). As a control, HEK-293T cells were co-transfected with the pMND-GFP lentiviral vector and four lentiviral packaging plasmids (pMD2.G, pRSV-rev, pMDLG-pRRE, and pRRL-GOI), and viral particles were harvested from the cell supernatant to produce lentiviral particles (293T control particles).

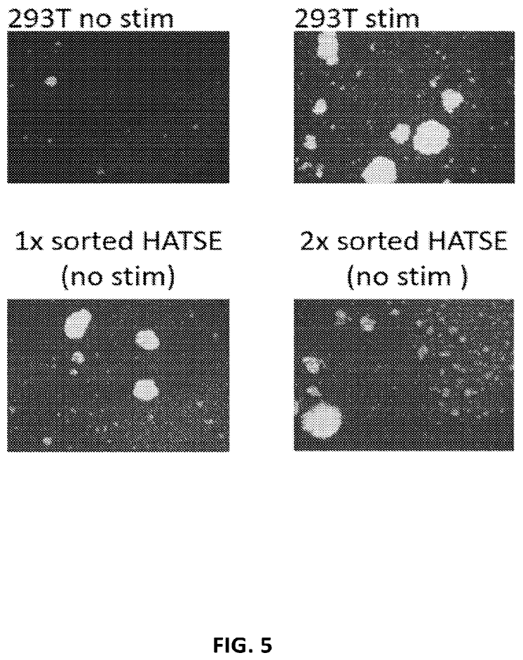

Human primary T cells (2.5×10⁵ cells) were exposed to either HATSE particles or 293T particles, at MOI 5 or MOI 20, in duplicate, in the presence or absence of exogenous T-cell activation stimulus (Dynabeads™ Human T-Activator CD3/CD28—“stimbeads”). Growth of cells was evaluated over 7 days by an automated cell counter (Countess™ II, Thermo Fisher) (FIG. 3). Growth of cells exposed to HATSE particles was similar to bead-stimulated T cells transduced with control particles, and was roughly two times greater at 7 days than 293T control particles. GFP expression was evaluated by fluorescence microscopy (FIG. 4). Cells exposed to HATSE particles exhibit blasting and show high GFP expression similar to bead-stimulated T cells transduced with control particles, confirming efficient transduction of the HATSE-particle exposed T-cells, despite the lack of exposure to a heterologous T-cell activating stimulus (i.e., the stimulating beads).

### Example 2: Lenti-RACCR

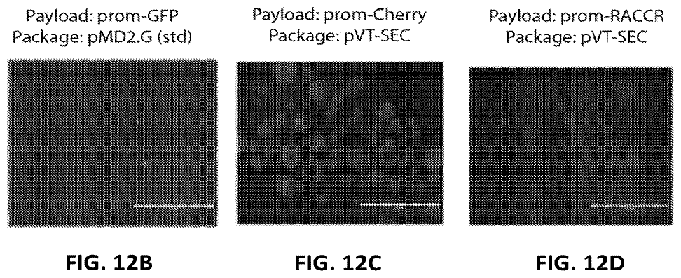

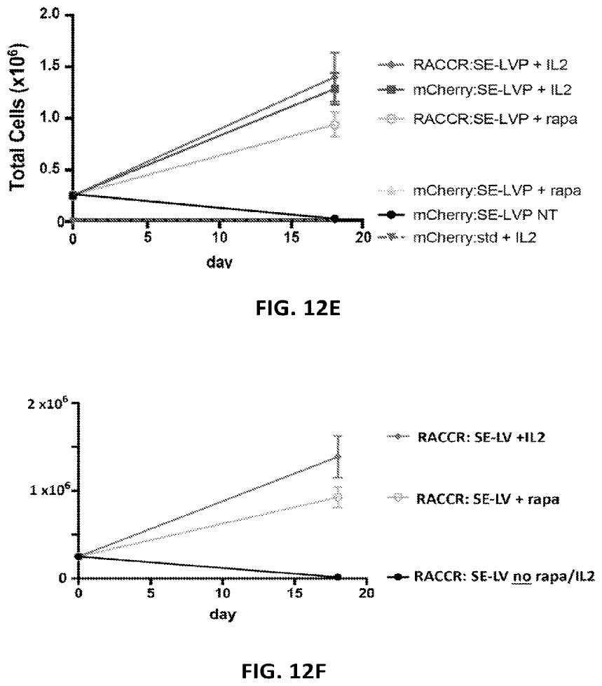

Lenti-RACCR is a lentiviral vector that contains a transgene encoding a rapamycin-activated chimeric cell-surface receptor (RACCR). Specifically this exemplary RACCR is made from two fusion proteins that are designed to dimerize in the presence of rapamycin, thereby activating signalling (FIG. 12A). The first fusion protein is the result of fusing the cytoplasmic domain of the IL-2 receptor beta chain (IL2Rb) to FK506 binding protein (FKBP) so that FKBP forms the extracellular domain of the fusion protein. The second fusion protein is the result of fusing the cytoplasmic domain of IL-2 receptor gamma chain (IL2Rg) to the FKBP-rapamycin-binding (FRB) domain of mammalian target of rapamycin (mTOR) so that FRB forms the extracellular domain of the fusion protein. The extracellular domains are known to form a trimeric complex in the presence of rapamycin, resulting in dimerization of the receptor subunits to form an actively signaling receptor complex.

RACCR-beta—FKBP-IL2Rb fusion protein with signal peptide (in parenthesis) with 2A peptide (in italics):

(SEQ ID NO: 12)
(MALPVTALLLPLALLLHAARP)ILWHEMWHEGLEEASRLYFGERNVKGMF
EVLEPLHAMMERGPQTLKETSFNQAYGRDLMEAQEWCRKYMKSGNVKDLLQ
AWDLYYHVFRRISKGKDTIPWLGHLLVGLSGAFGFIILVYLLINCRNTGPW
LKKVLKCNTPDPSKFFSQLSSEHGGDVQKWLSSPFPSSSFSPGGLAPEISP
LEVLERDKVTQLLLQQDKVPEPASLSSNHSLTSCFTNQGYFFFHLPDALEI
EACQVYFTYDPYSEEDPDEGVAGAPTGSSPQPLQPLSGEDDAYCTFPSRDD
LLLFSPSLLGGPSPPSTAPGGSGAGEERMPPSLQERVPRDWDPQPLGPPTP
GVPDLVDFQPPPELVLREAGEEVPDAGPREGVSFPWSRPPGQGEFRALNAR
LPLNTDAYLSLQELQGQDPTHLVGSGATNFSLLKQAGDVEENPG.

The FKBP domain has the following sequence:

(SEQ ID NO: 13)
EMWHEGLEEASRLYFGERNVKGMFEVLEPLHAMMERGPQTLKETSFNQAYG
RDLMEAQEWCRKYMKSGNVKDLLQAWDLYYHVFRRISK.

The IL2Rb domain has the following sequence:

(SEQ ID NO: 14)
GKDTIPWLGHLLVGLSGAFGFIILVYLLINCRNTGPWLKKVLKCNTPDPSK
FFSQLSSEHGGDVQKWLSSPFPSSSFSPGGLAPEISPLEVLERDKVTQLLL
QQDKVPEPASLSSNHSLTSCFTNQGYFFFHLPDALEIEACQVYFTYDPYSE
EDPDEGVAGAPTGSSPQPLQPLSGEDDAYCTFPSRDDLLLFSPSLLGGPSP
PSTAPGGSGAGEERMPPSLQERVPRDWDPQPLGPPTPGVPDLVDFQPPPEL
VLREAGEEVPDAGPREGVSFPWSRPPGQGEFRALNARLPLNTDAYLSLQEL
QGQDPTHLV.

RACCR-gamma—FRB-IL2Rg fusion protein with signal peptide (in parenthesis) with 2A peptide (in italics)

(SEQ ID NO: 15)
(MPLGLLWLGLALLGALHAQA)GVQVETISPGDGRTFPKRGQTCVVHYTGM
LEDGKKFDSSRDRNKPFKFMLGKQEVIRGWEEGVAQMSVGQRAKLTISPDY
AYGATGHPGIIPPHATLVFDVELLKLGEGSNTSKENPFLFALEAVVISVGS
MGLIISLLCVYFWLERTMPRIPTLKNLEDLVTEYHGNFSAWSGVSKGLAES
LQPDYSERLCLVSEIPPKGGALGEGPGASPCNQHSPYWAPPCYTLKPETGS
GATNFSLLKQAGDVEENPG.

The FRB domain has the following sequence:

(SEQ ID NO: 16)
GVQVETISPGDGRTFPKRGQTCVVHYTGMLEDGKKFDSSRDRNKPFKFMLG
KQEVIRGWEEGVAQMSVGQRAKLTISPDYAYGATGHPGIIPPHATLVFDVE
LLKLGE.

The IL2Rg domain has the following sequence:

(SEQ ID NO: 17)
GSNTSKENPFLFALEAVVISVGSMGLIISLLCVYFWLERTMPRIPTLKNLE
DLVTEYHGNFSAWSGVSKGLAESLQPDYSERLCLVSEIPPKGGALGEGPGA
SPCNQHSPYWAPPCYTLKPET.

A lentiviral transfer plasmid encoding green fluorescence protein is packaged into lentiviral particles using the standard packaging plasmid pMD2.G, and the resulting lentiviral particles are used to transduce human primary T cells as a control experiment. Only low-level transduction is observed (FIG. 12B).

A lentiviral transfer plasmid encoding mCherry is packaged into lentiviral particles using a plasmid encoding T-cell activation and co-stimulatory molecules, thereby generating a surface-engineered lentiviral particle (SE-LVP). The resulting lentiviral particles (mCherry: SE-LVP) are used to transduce human primary T cells as a control experiment. High-level transduction is observed (FIG. 12C).

A lentiviral transfer plasmid encoding RACCR is packaged into lentiviral particles using a plasmid encoding T-cell activation and co-stimulatory molecules, thereby generating a surface-engineered lentiviral particle (SE-LVP). The resulting lentiviral particles (RACCR: SE-LVP) are used to transduce human primary T cells. High-level transduction is observed (FIG. 12D).

Human primary T-cell transduced with mCherry: SE-LVP or RACCR: SE-LVP are cultured in the presence of IL-2 (“IL2”), rapamycin (“rapa”), or no treatment (“NT”). As a control the mCherry contstruct packaged using the standard packaging plasmid pMD2.G is also tested with IL-2. RACCR: SE-LVP mediates sustained expansion of T-cell over 18 days in the presence of rapamycin but without exogenous IL-2 (FIG. 12E). T-cell expansion is dependent on the presence of rapamycin or IL-2 (FIG. 12F). This constitutes in vitro proof of concept that surface engineered particles can deliver a growth-stimulatory receptor payload and expand primary human T-cells. No spinoculation or other manipulations of the lentiviral particles is required for transduction. Transduction of cells at a multiplicity of infection (MOI) of 10 provided approximately 20% transduction efficiency.

Those skilled in the art will recognize, or be able to ascertain using no more than routine experimentation, many equivalents to the specific embodiments of the invention described herein. Such equivalents are intended to be encompassed by the following claims.

What is claimed:

1. A lentiviral particle for activating and efficiently transducing T cells, comprising a nucleic acid sequence encoding a small molecule controllable T-cell/NK-cell activation receptor, the nucleic acid sequence operatively linked to a promoter, wherein the T-cell/NK-cell activation receptor is capable of being activated by the small molecule;
wherein the T-cell/NK-cell activation receptor comprises two polypeptide chains that comprise extracellular domains that bind the small molecule, and wherein the two polypeptide chains each comprise a transmembrane domain and an intracellular cytokine receptor signaling domain; and
wherein the lentiviral particle further comprises a viral surface comprising a CD28 ligand.

2. The lentiviral particle of claim 1, wherein the lentiviral particle is a surface-engineered lentiviral particle further comprising a T-cell activation or co-stimulation molecule.

3. The lentiviral particle of claim 2, wherein the T-cell activation or co-stimulation molecule comprises a 41bb ligand.

4. The lentiviral particle of claim 1, wherein the intracellular cytokine receptor signaling domain of a first polypeptide chain of the two polypeptide chains comprises a cytokine receptor signaling domain of a common cytokine receptor gamma chain.

5. The lentiviral particle of claim 4, wherein the intracellular cytokine receptor signaling domain of a second polypeptide chain of the two polypeptide chains comprises a cytokine receptor signaling domain of a common cytokine receptor beta chain.

6. The lentiviral particle of claim 1, wherein the extracellular domains of the T-cell/NK-cell activation receptor comprise a FK506 binding protein (FKBP) or a functional homolog thereof.

7. The lentiviral particle of claim 1, wherein the extracellular domains of the T-cell/NK-cell activation receptor comprise a FKBP12-rapamycin binding (FRB) protein or a functional homolog thereof.

8. The lentiviral particle of claim 1, wherein the lentiviral particle further comprises a nucleic acid sequence encoding a checkpoint-inhibiting ligand.

9. The lentiviral particle of claim 8, wherein the checkpoint-inhibiting ligand is capable of blocking a PD-1/PD-L1 checkpoint or a Tim-3 checkpoint.

10. The lentiviral particle of claim 1, further comprising a nucleic acid sequence encoding a protein that provides resistance to an immunosuppressive drug.

11. The lentiviral particle of claim 10, wherein the immunosuppressive drug is selected from the group consisting of methotrexate, rapamycin, a rapalog, tacrolimus, and cyclosporine.

12. The lentiviral particle of claim 1, further comprising a nucleic acid sequence encoding a TGFbeta dominant-negative inhibiting receptor.

13. The lentiviral particle of claim 1, wherein the sequence of the promoter is at least 95% identical to a sequence selected from the group consisting of SEQ ID NOs: 1-5.

14. A method for expanding T-cells capable of recognizing and killing tumor cells in a subject in need thereof, comprising:
i) administering a lentiviral particle of claim 1 to the subject, and
ii) administering the small molecule to the subject,
wherein T-cells capable of recognizing and killing tumor cells in the subject are expanded.

15. The lentiviral particle of claim 1, wherein the CD28 ligand comprises a CD80 protein.

16. The lentiviral particle of claim 1, wherein the CD28 ligand comprises a CD86 protein.

17. The lentiviral particle of claim 1, wherein binding of the extracellular domains to the small molecule is sufficient for the intracellular cytokine receptor signaling domains of the two polypeptide chains to activate cytokine signal transduction.

18. The lentiviral particle of claim 1, wherein the T-cell/NK-cell activation receptor does not comprise an antigen binding domain.

19. The lentiviral particle of claim 1, wherein the small molecule is rapamycin.

20. The lentiviral particle of claim 1, wherein the small molecule comprises rapamycin, a rapalog, tacrolimus, coumermycin, gibberellin, abscisic acid, methotrexate, cyclosporin A, FKCsA, trimethoprim-synthetic ligand for FKBP, or derivatives thereof.


SEQUENCE LISTING (17 sequences)

SEQ ID NO: 1 - DNA, 1485 bp, synthetic construct
  misc_feature 1..1485: note = TCP1 - murine Tce1 core enhancer with Dap10 minimal promoter
  source 1..1485: mol_type = other DNA
gtgcacagtccaggcaggtctccagcttgcaaacctcctgactcagtggctgggattacagatttgtgtgagcagcaggcatagatttttctttaaaaaaaataaaataaaataaaaaataaagcccagtgttttttcacatggtctgggaagggcctaacaagtgggtttcctgcagaggaagaggagagcgttgccctgactctcacctgctggagggggtagagaggggcctgcagccaagtcatcatttttgaatgtgtgtcagttacaactacaaaggcaaccaaaaggaggtcttatccgttgtctgtgagcttcagttgggaacagcaggcagcatttaggtttggggttttttttgttgtttttttttcacccccaaagctataaatagcccagcatgtggggcatcctggctgctctaccttacacttgagatccaagcaggtagccacccccttctcccctgcagacattttccacaacacgtatgtgatgagaagaaatgtctcccggagtggggtgtgtgtgtgtattcgtatgttgttattttggtcttaactttgatgcatgccacccacgctctcagccaggggtgcgagtcctccttgatgccttaacgaagtggtgggcttcgaaggaagagaggaaggaagaaaacaactctactgtccagatatgctcttactttaagtttttgttacttaaagtttgttgatagtctataaactgactttgttctatgtgtctctaggaagctatgctttctgcagattttcttaactacctcccaactctttggtgaagaaaatggtctttactctttgccctttatgagtgagtgtgtgtgtgtgtgtgtgtgtgtgtgtgtgtgtgtatgcaacagttgttgtccttgcttattctccatcatttttttccacatagactagctggcgaatgagctgcaggatttcaagtctttctaccctgaccccagtgccggggttacggacatgcctgcctgcaacatgaggacttttcaagtggacgctgagggcgctgatctcaggcccttgtgttctcgtggaaaacactttacaaacagaaccatctcccaagcctgtcttctcacagttcatgaactcaggggcgtgtctctctgtggttagactatgaagccactgtgtcaacgccattctcctcgagttttcttggccctacctccaaaatatttccagatctccctagcctgcacacccttgccacctgtcattcccacttggaccaggccagcagcctccctggtctctctgaccctccccctgagttcgttcaccaaaggcagtaacggagacaccccctcaacacacacaggaagcagatggccttgacaccagcagggtgacatccgctattgctacttctctgctcccccacagttcctctggacttctctggaccacagtcctctgccagacccctgccagaccccagtccacc

SEQ ID NO: 2 - DNA, 1540 bp, synthetic construct
  misc_feature 1..1540: note = TCP1 - murine Tce1 core enhancer with Dap10 minimal promoter
  source 1..1540: mol_type = other DNA
cacaatactttggtataaatttgggattaattcgaaagttcttgattgtaaataataaactttccccaagaaaaggagtacgatgaactttctttaagaaaaaaaaaaaaaatccaatgcatttcacatgaccaaggattgcacagatgcatttctggaccaaggaggagaagattgaacgttggaggggggctgcccgtctgacttcctcacctgttcgagggggtcgggaaggggggttaccgctaagtcatcacttttttatgtgtgtcggttaaaactacaaaggcaaccaaaaggaggtcttatccgttttttgtgagcttcagttgggagcagcaggtagaagaagctgcactctggttttactttttcaccaccgggacctataaatagatgaggctgtgtggctttctcgctgagttacctccaccttcttgcctgggacctcacgatccaggcggctctaactcccacatgaacattcgcagctcactgcccccgcagtcattttccacaacatgtgtgtgacatgaagaagtgcgtctcagcatggtgtgtttgtgtgtgttgttgttatttttgttttagcgttgagttctagatgtcttccgcccacactgccaactctggaagcaaggctttctaaatgtctcgaagaggtcctcgatatttgttgaaggaagaaaacattgctcttggttatttttgctccccatgtatccaaaaatacactaacttttatgtttcataactattcaatactagtccctcggccgctcttacatgatgatgtacagtgtttatctgccaggccgcgttttgtacaaactgcctttactctccccaactatttggtaaagaaaattattttcactttttttgttgttgttgtttgtttttgtttgttttgagagggaatattgctctgttgccaggctggagtgcagtggagtgatctcggcttactgcaacctccacctcccgggttcaagcgattctcctgcctcagcctcctgagtcactgggattacaggcgtgcgccaacacacacagctaatttttgtatttttagtagagctggggtttcaccatgttggccaggatgatctcaatctcctgacctcgtgatctgcccgcttcagcctcccaaagtgctgggattacaggcgtgagccaccgcgcccggcctattttcacttttagtaaatggaaatctgggtatcaagagaagtcagtttctattttttcttggccctacctccaaaatatttccagatctccctagcctgcacacccttgccacctgtcattcccacttggaccaggccagcagcctccctggtctctctgaccctccccctgagttcgttcaccaaaggcagtaacggagacaccccctcaacacacacaggaagcagatggccttgacaccagcagggtgacatccgctattgctacttctctgctcccccacagttcctctggacttctctggaccacagtcctctgccagacccctgccagaccccagtccacc

SEQ ID NO: 3 - DNA, 2416 bp, synthetic construct
  misc_feature 1..2416: note = TCP3 - full CD2 enhancer with Dap10 minimal promoter
  source 1..2416: mol_type = other DNA
agctttgactagacccgtgtctgctcattgtgtgaaggatcccagtgtcgaggagaggaagctgccgaataagaaagagcctcgctggtgtgacagcaggatcaaattcagtcaactcaatccggctgagacctccttagtcctcaccagaatgacccgaacatgctccccaggtctcagctgcaaagctgggtccaacaacctatttgtagatttccataatgaattaaagcctccatgcacaaacgttaacaggagtcaccagaaaacttaacattttaggtcttactttcttgactgttcagacacggtcataatgacctgtactgtcatccttcaaaacaagggaatctatggatactggaggtgtcatgaactcatcagatgacatacaaaaagataggctaagagacaactcagttgaatcaaatcttaagtgtcttaaatgactatgattaaaaggtcagaccccatccccagctagcagtcatgcagctgtactttgagggaagtggggagaaaaaaatagttttcagttagagttctaaaacaataggtggtgagaaggcacctgctttgaaatgctggtgccttgtctgtggtcctgtctgcccgccttctccttggcttcttagacttgctaaaccttgttacactctggcaggaagctctgagggctaaaccacaaaaactaagtacattcaggacttcctgtgacagctcactgaggattgaactactttttcctctgaatctgagagtctatttttccagcaaccagagttaattactaagggcaatgatgaatgaaacctcatcgcctcccaaaattcaaacacaaaatccagctaggtaacaattgccacaggatctacccagtagagagatgagggctctggccgggcatggtggctcgtgccagtaatcctaacactttgggaggccaaagcaggtagatcacctgaggtcaggagtttgagaccagcctggccaacatggtgaaaccccttctctacttaaaaaaaaaaaaaaaaaaattagccgggcatcgtggcacgcacctataaccccagctactcgggaggttgaggcagaataattgcttgaaccctggggtgtggaggttgcagtgaggtgagatcgtgtcacttcgctctagcctgggcaaaagagcaaaactccgtctaaaaaaaaaaaggagagagagagagagagagagagagagatgagggtcctagctctgcccctttctcattcttcatgatacccatgagtgatttcattggctatctcaccagaacagctcatacgccattcccttaagaaaatcttccctggtctattcagccctgggctctgcctttcttctgaactcctgtggcattacatatggtacaaaacacatacaccctattcaatccttcatcgttcattcattcaacgaacatttattgaacttctactatgtgtcaggctctgtgctaggcactaggaaaacagaggtgtacaattgcatctctgctttcaaggagctcaccacctggtaggcaagaaacggaccaaccatgggacaaatgcaatgcagtatgataaatataggtcagaggcaagtgcatgaacccagaggaaggactttgaacccagactgagtggtaagactggggtgacaaagagggcttcctagaggagggacgtttgggttgcgttgtaatagattaattgaactggtgaagcagaggtacagaattacccatgtagaggcaacagcatgagcaaggctcatttggctttctgtttacacaaacatgtagtttctttttcaactactgtatttaactgatttgaagatgtcattgatttttcaactactgttttcaactttttgtatttctttttcttttttaacaactatatttaactaatttgaagatggcattgattttaagtggcatcattcttttacatgccactaagaaagagaagaacacttctcagttgcattgtaacaaatactaagatacagtcaactttcagtcacaatcccattcctgagatattaaaatgtgaaaaaatgttattttatgttagtgcattttagaattgatgaaatgcatagtaaagattttcttggccctacctccaaaatatttccagatctccctagcctgcacacccttgccacctgtcattcccacttggaccaggccagcagcctccctggtctctctgaccctccccctgagttcgttcaccaaaggcagtaacggagacaccccctcaacacacacaggaagcagatggccttgacaccagcagggtgacatccgctattgctacttctctgctcccccacagttcctctggacttctctggaccacagtcctctgccagacccctgccagaccccagtccacc

SEQ ID NO: 4 - DNA, 824 bp, synthetic construct
  misc_feature 1..824: note = TCP4 - full CD2 enhancer with Dap10 minimal promoter
  source 1..824: mol_type = other DNA
ttaagtgtcttaaatgactatgattaaaaggtcagaccccatccccagctagcagtcatgcagctgtactttgagggaagtggggagaaaaaaatagttttcagttagagttctaaaacaataggtggtgagaaggcacctgctttgaaatgctggtgccttgtctgtggtcctgtctgcccgccttctccttggcttcttagacttgctaaaccttgttacactctggcaggaagctctgagggctaaaccacaaaaactaagtacattcaggacttcctgtgacagctcactgaggattgaactactttttcctctgaatctgagagtctatttttccagcaaccagagttaattactaagggcaatgatgaatgaaacctcatcgcctcccaaaattcaaacacaaaatccagctaggtaacaattgccacaggatctacccagtagagagatgagggctctggccgggcatggtggctcgtgccagtaatcctaacactttgggaggccaaagcaggtagatcaccttttcttggccctacctccaaaatatttccagatctccctagcctgcacacccttgccacctgtcattcccacttggaccaggccagcagcctccctggtctctctgaccctccccctgagttcgttcaccaaaggcagtaacggagacaccccctcaacacacacaggaagcagatggccttgacaccagcagggtgacatccgctattgctacttctctgctcccccacagttcctctggacttctctggaccacagtcctctgccagacccctgccagaccccagtccacc

SEQ ID NO: 5 - DNA, 347 bp, synthetic construct
  misc_feature 1..347: note = MND Promoter
  source 1..347: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 6 - DNA, 9859 bp, synthetic construct
  misc_feature 1..9859: note = lentiviral vector vivo-TIL 104 - LNGFR
  source 1..9859: mol_type = other DNA
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggacgcgtaggaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgctagcaccggtcctgcagggccgccaccatgcctctgggcctgctgtggctgggcctggccctgctgggcgccctgcacgcccaggccggcgtgcaggtggagacaatctccccaggcgacggacgcacattccctaagcggggccagacctgcgttgtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagggcagcggcgccaccaacttcagcctgctgaagcaggccggcgacgtggaggagaaccccggccccggtgctggcgcaactggacgcgctatggatggacctcgcttgctgcttcttctgcttctcggggtctctttgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcctgtcaggataagcagaacaccgtctgcgaagagtgccccgatggtacctacagcgatgaagcgaaccatgtagacccatgcctgccttgcaccgtttgtgaagacacggaacgacagttgcgggaatgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcgtacatcgctttcaaaagatgaacgccggcgctagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 7 - DNA, 10997 bp, synthetic construct
  misc_feature 1..10997: note = lentiviral vector vivo-TIL 105 - TCP1
  source 1..10997: mol_type = other DNA
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggacgcgtaggtgcacagtccaggcaggtctccagcttgcaaacctcctgactcagtggctgggattacagatttgtgtgagcagcaggcatagatttttctttaaaaaaaataaaataaaataaaaaataaagcccagtgttttttcacatggtctgggaagggcctaacaagtgggtttcctgcagaggaagaggagagcgttgccctgactctcacctgctggagggggtagagaggggcctgcagccaagtcatcatttttgaatgtgtgtcagttacaactacaaaggcaaccaaaaggaggtcttatccgttgtctgtgagcttcagttgggaacagcaggcagcatttaggtttggggttttttttgttgtttttttttcacccccaaagctataaatagcccagcatgtggggcatcctggctgctctaccttacacttgagatccaagcaggtagccacccccttctcccctgcagacattttccacaacacgtatgtgatgagaagaaatgtctcccggagtggggtgtgtgtgtgtattcgtatgttgttattttggtcttaactttgatgcatgccacccacgctctcagccaggggtgcgagtcctccttgatgccttaacgaagtggtgggcttcgaaggaagagaggaaggaagaaaacaactctactgtccagatatgctcttactttaagtttttgttacttaaagtttgttgatagtctataaactgactttgttctatgtgtctctaggaagctatgctttctgcagattttcttaactacctcccaactctttggtgaagaaaatggtctttactctttgccctttatgagtgagtgtgtgtgtgtgtgtgtgtgtgtgtgtgtgtgtgtatgcaacagttgttgtccttgcttattctccatcatttttttccacatagactagctggcgaatgagctgcaggatttcaagtctttctaccctgaccccagtgccggggttacggacatgcctgcctgcaacatgaggacttttcaagtggacgctgagggcgctgatctcaggcccttgtgttctcgtggaaaacactttacaaacagaaccatctcccaagcctgtcttctcacagttcatgaactcaggggcgtgtctctctgtggttagactatgaagccactgtgtcaacgccattctcctcgagttttcttggccctacctccaaaatatttccagatctccctagcctgcacacccttgccacctgtcattcccacttggaccaggccagcagcctccctggtctctctgaccctccccctgagttcgttcaccaaaggcagtaacggagacaccccctcaacacacacaggaagcagatggccttgacaccagcagggtgacatccgctattgctacttctctgctcccccacagttcctctggacttctctggaccacagtcctctgccagacccctgccagaccccagtccaccgctagcaccggtcctgcagggccgccaccatgcctctgggcctgctgtggctgggcctggccctgctgggcgccctgcacgcccaggccggcgtgcaggtggagacaatctccccaggcgacggacgcacattccctaagcggggccagacctgcgttgtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagggcagcggcgccaccaacttcagcctgctgaagcaggccggcgacgtggaggagaaccccggccccggtgctggcgcaactggacgcgctatggatggacctcgcttgctgcttcttctgcttctcggggtctctttgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcctgtcaggataagcagaacaccgtctgcgaagagtgccccgatggtacctacagcgatgaagcgaaccatgtagacccatgcctgccttgcaccgtttgtgaagacacggaacgacagttgcgggaatgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcgtacatcgctttcaaaagatgacgccggcgactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 8 - DNA, 11035 bp, synthetic construct
  misc_feature 1..11035: note = lentiviral vector vivo-TIL 106 - TCP2
  source 1..11035: mol_type = other DNA
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggacgcgtagacaatactttggtataaatttgggattaattcgaaagttcttgattgtaaataataaactttccccaagaaaaggagtacgatgaactttctttaagaaaaaaaaaaaaaatccaatgcatttcacatgaccaaggattgcacagatgcatttctggaccaaggaggagaagattgaacgttggaggggggctgcccgtctgacttcctcacctgttcgagggggtcgggaaggggggttaccgctaagtcatcacttttttatgtgtgtcggttaaaactacaaaggcaaccaaaaggaggtcttatccgttttttgtgagcttcagttgggagcagcaggtagaagaagctgcactctggttttactttttcaccaccgggacctataaatagatgaggctgtgtggctttctcgctgagttacctccaccttcttgcctgggacctcacgatccaggcggctctaactcccacatgaacattcgcagctcactgcccccgcagtcattttccacaacatgtgtgtgacatgaagaagtgcgtctcagcatggtgtgtttgtgtgtgttgttgttatttttgttttagcgttgagttctagatgtcttccgcccacactgccaactctggaagcaaggctttctaaatgtctcgaagaggtcctcgatatttgttgaaggaagaaaacattgctcttggttatttttgctccccatgtatccaaaaatacactaacttttatgtttcataactattcaatactagtccctcggccgctcttacatgatgatgtacagtgtttatctgccaggccgcgttttgtacaaactgcctttactctccccaactatttggtaaagaaaattattttcactttttttgttgttgttgtttgtttttgtttgttttgagagggaatattgctctgttgccaggctggagtgcagtggagtgatctcggcttactgcaacctccacctcccgggttcaagcgattctcctgcctcagcctcctgagtcactgggattacaggcgtgcgccaacacacacagctaatttttgtatttttagtagagctggggtttcaccatgttggccaggatgatctcaatctcctgacctcgtgatctgcccgcttcagcctcccaaagtgctgggattacaggcgtgagccaccgcgcccggcctattttcacttttagtaaatggaaatctgggtatcaagagaagtcagtttctattttttcttggccctacctccaaaatatttccagatctccctagcctgcacacccttgccacctgtcattcccacttggaccaggccagcagcctccctggtctctctgaccctccccctgagttcgttcaccaaaggcagtaacggagacaccccctcaacacacacaggaagcagatggccttgacaccagcagggtgacatccgctattgctacttctctgctcccccacagttcctctggacttctctggaccacagtcctctgccagacccctgccagaccccagtccaccgctagcaccggtgccgccaccatgcctctgggcctgctgtggctgggcctggccctgctgggcgccctgcacgcccaggccggcgtgcaggtggagacaatctccccaggcgacggacgcacattccctaagcggggccagacctgcgttgtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagggcagcggcgccaccaacttcagcctgctgaagcaggccggcgacgtggaggagaaccccggccccggtgctggcgcaactggacgcgctatggatggacctcgcttgctgcttcttctgcttctcggggtctctttgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcctgtcaggataagcagaacaccgtctgcgaagagtgccccgatggtacctacagcgatgaagcgaaccatgtagacccatgcctgccttgcaccgtttgtgaagacacggaacgacagttgcgggaatgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcgtacatcgctttcaaaagatgaactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 9 - DNA, 11912 bp, synthetic construct
  misc_feature 1..11912: note = lentiviral vector vivo-TIL 107 - TCP3
  source 1..11912: mol_type = other DNA
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggacgcgtagagctttgactagacccgtgtctgctcattgtgtgaaggatcccagtgtcgaggagaggaagctgccgaataagaaagagcctcgctggtgtgacagcaggatcaaattcagtcaactcaatccggctgagacctccttagtcctcaccagaatgacccgaacatgctccccaggtctcagctgcaaagctgggtccaacaacctatttgtagatttccataatgaattaaagcctccatgcacaaacgttaacaggagtcaccagaaaacttaacattttaggtcttactttcttgactgttcagacacggtcataatgacctgtactgtcatccttcaaaacaagggaatctatggatactggaggtgtcatgaactcatcagatgacatacaaaaagataggctaagagacaactcagttgaatcaaatcttaagtgtcttaaatgactatgattaaaaggtcagaccccatccccagctagcagtcatgcagctgtactttgagggaagtggggagaaaaaaatagttttcagttagagttctaaaacaataggtggtgagaaggcacctgctttgaaatgctggtgccttgtctgtggtcctgtctgcccgccttctccttggcttcttagacttgctaaaccttgttacactctggcaggaagctctgagggctaaaccacaaaaactaagtacattcaggacttcctgtgacagctcactgaggattgaactactttttcctctgaatctgagagtctatttttccagcaaccagagttaattactaagggcaatgatgaatgaaacctcatcgcctcccaaaattcaaacacaaaatccagctaggtaacaattgccacaggatctacccagtagagagatgagggctctggccgggcatggtggctcgtgccagtaatcctaacactttgggaggccaaagcaggtagatcacctgaggtcaggagtttgagaccagcctggccaacatggtgaaaccccttctctacttaaaaaaaaaaaaaaaaaaattagccgggcatcgtggcacgcacctataaccccagctactcgggaggttgaggcagaataattgcttgaaccctggggtgtggaggttgcagtgaggtgagatcgtgtcacttcgctctagcctgggcaaaagagcaaaactccgtctaaaaaaaaaaaggagagagagagagagagagagagagagatgagggtcctagctctgcccctttctcattcttcatgatacccatgagtgatttcattggctatctcaccagaacagctcatacgccattcccttaagaaaatcttccctggtctattcagccctgggctctgcctttcttctgaactcctgtggcattacatatggtacaaaacacatacaccctattcaatccttcatcgttcattcattcaacgaacatttattgaacttctactatgtgtcaggctctgtgctaggcactaggaaaacagaggtgtacaattgcatctctgctttcaaggagctcaccacctggtaggcaagaaacggaccaaccatgggacaaatgcaatgcagtatgataaatataggtcagaggcaagtgcatgaacccagaggaaggactttgaacccagactgagtggtaagactggggtgacaaagagggcttcctagaggagggacgtttgggttgcgttgtaatagattaattgaactggtgaagcagaggtacagaattacccatgtagaggcaacagcatgagcaaggctcatttggctttctgtttacacaaacatgtagtttctttttcaactactgtatttaactgatttgaagatgtcattgatttttcaactactgttttcaactttttgtatttctttttcttttttaacaactatatttaactaatttgaagatggcattgattttaagtggcatcattcttttacatgccactaagaaagagaagaacacttctcagttgcattgtaacaaatactaagatacagtcaactttcagtcacaatcccattcctgagatattaaaatgtgaaaaaatgttattttatgttagtgcattttagaattgatgaaatgcatagtaaagattttcttggccctacctccaaaatatttccagatctccctagcctgcacacccttgccacctgtcattcccacttggaccaggccagcagcctccctggtctctctgaccctccccctgagttcgttcaccaaaggcagtaacggagacaccccctcaacacacacaggaagcagatggccttgacaccagcagggtgacatccgctattgctacttctctgctcccccacagttcctctggacttctctggaccacagtcctctgccagacccctgccagaccccagtccaccgctagcaccggtgccgccaccatgcctctgggcctgctgtggctgggcctggccctgctgggcgccctgcacgcccaggccggcgtgcaggtggagacaatctccccaggcgacggacgcacattccctaagcggggccagacctgcgttgtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagggcagcggcgccaccaacttcagcctgctgaagcaggccggcgacgtggaggagaaccccggccccggtgctggcgcaactggacgcgctatggatggacctcgcttgctgcttcttctgcttctcggggtctctttgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcctgtcaggataagcagaacaccgtctgcgaagagtgccccgatggtacctacagcgatgaagcgaaccatgtagacccatgcctgccttgcaccgtttgtgaagacacggaacgacagttgcgggaatgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcgtacatcgctttcaaaagatgaactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 10 - DNA, 10320 bp, synthetic construct
  misc_feature 1..10320: note = lentiviral vector vivo-TIL 108 - TCP4
  source 1..10320: mol_type = other DNA
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggacgcgtagttaagtgtcttaaatgactatgattaaaaggtcagaccccatccccagctagcagtcatgcagctgtactttgagggaagtggggagaaaaaaatagttttcagttagagttctaaaacaataggtggtgagaaggcacctgctttgaaatgctggtgccttgtctgtggtcctgtctgcccgccttctccttggcttcttagacttgctaaaccttgttacactctggcaggaagctctgagggctaaaccacaaaaactaagtacattcaggacttcctgtgacagctcactgaggattgaactactttttcctctgaatctgagagtctatttttccagcaaccagagttaattactaagggcaatgatgaatgaaacctcatcgcctcccaaaattcaaacacaaaatccagctaggtaacaattgccacaggatctacccagtagagagatgagggctctggccgggcatggtggctcgtgccagtaatcctaacactttgggaggccaaagcaggtagatcaccttttcttggccctacctccaaaatatttccagatctccctagcctgcacacccttgccacctgtcattcccacttggaccaggccagcagcctccctggtctctctgaccctccccctgagttcgttcaccaaaggcagtaacggagacaccccctcaacacacacaggaagcagatggccttgacaccagcagggtgacatccgctattgctacttctctgctcccccacagttcctctggacttctctggaccacagtcctctgccagacccctgccagaccccagtccaccgctagcaccggtgccgccaccatgcctctgggcctgctgtggctgggcctggccctgctgggcgccctgcacgcccaggccggcgtgcaggtggagacaatctccccaggcgacggacgcacattccctaagcggggccagacctgcgttgtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagggcagcggcgccaccaacttcagcctgctgaagcaggccggcgacgtggaggagaaccccggccccggtgctggcgcaactggacgcgctatggatggacctcgcttgctgcttcttctgcttctcggggtctctttgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcctgtcaggataagcagaacaccgtctgcgaagagtgccccgatggtacctacagcgatgaagcgaaccatgtagacccatgcctgccttgcaccgtttgtgaagacacggaacgacagttgcgggaatgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcgtacatcgctttcaaaagatgaactagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 11 - DNA, 10949 bp, synthetic construct
  misc_feature 1..10949: note = lentiviral vector vivo-TIL 109 - hPerfP
  source 1..10949: mol_type = other DNA
agcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacaataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggacgcgtagttccaaagtcctctctttgattttataggtgaggaaactaacgctcagaaagggggttgatatctatcgccgtgaggcatacggtaagtttctggtgaagctgggatcagaacctgtttagactttgcctctcttttcccgcagatactttgcaggacttctatgtccctcaaaccggccttcctgtcatggtcaggaaagaaactcctcacagcctcagcatccaagtcaggccatgggtgacagctggaaagtgatcaggaggctgcagtttctagaagagggtggggacactgcggagagaagatggggccagattccgagaagacagcataagcccctgttcctgtaagagcagggacggaagcagggacataaacgcaagggatgagccccaaagtgtgacccatgagacatgatgtcacatgtggtctggagcctgcggccacttcttcccatcatatacacagttatgagaacaagttgtgagaaccacctcctcccttacccagctgcccccaccccagaagccgtgtgattttgccccccagtgccctgtgagtcactccacccatggaaacctcaccccaccctgacctcaagcaaggcagagtgcagaagacatgtcctccggtgctaccagaccactctcaccagcacccacgacctcagcagggctggagccagcgtggaggccactggctgtcctcacaaagcgaggagcaggagcccctgttcgaggaacatgcttggagttcggagcctgggctagggtgggatgtaggttgagcaggaagtggatggcaagattagagcaacatctctcttctcccactcagggaggagggaatggccacaggctctgacactcaagaagggccaggcacagttccaagcacttcacaacaacccctagggtctacatgacctacaatcccaattgttcagtgaagaaactgaggcacagtgaggctgaagaaccctaccagtccacactgctggtgcataaccgagctgcccaagccccggcggtctggcgtgtaggcccatgctctgagccgccgcctctgcttgcctcttacatcccacacatgcgatgctgtgcatcagaagcaaggagatggccctgctggcctgttcatcaacaccagggccgagtctcaaagtcctcagcgccccgccctcctccgcctgtgtgccctgagtccccgagccccagcagctctactcggcagatgagcctctggccctgctgctcgcttcctgagggctgtcagtggggagccggatgagggctgaggacagggtgggtgcttgtgggaggggagagcacaaaggacctgtgaccacagctgggggcggggcaggaagtagaagtgatgtgagtggtggctggtgcaaggagccacagtgggctgcctggggggctgatgccacgctagcaccggtcctgcagggccgccaccatgcctctgggcctgctgtggctgggcctggccctgctgggcgccctgcacgcccaggccggcgtgcaggtggagacaatctccccaggcgacggacgcacattccctaagcggggccagacctgcgttgtgcactatacaggcatgctggaggatggcaagaagtttgacagctcccgggatagaaacaagccattcaagtttatgctgggcaagcaggaagtgatcagaggctgggaggagggcgtggcccagatgtctgtgggccagagggccaagctgaccatcagcccagactacgcctatggagcaacaggccacccaggaatcatcccacctcacgccaccctggtgttcgatgtggagctgctgaagctgggcgagggatccaacacatcaaaagagaacccctttctgttcgcattggaggccgtagtcatatctgttggatccatgggacttattatctccctgttgtgtgtgtacttctggctggaacggactatgcccaggatccccacgctcaagaatctggaagatctcgtcacagaataccatggtaatttcagcgcctggagcggagtctctaagggtctggccgaatccctccaacccgattattctgaacggttgtgcctcgtatccgaaataccaccaaaaggcggggctctgggtgagggcccaggggcgagtccgtgcaatcaacacagcccgtattgggcccctccttgttatacgttgaagcccgaaactggaagcggagctactaacttcagcctgctgaagcaggctggagacgtggaggagaaccctggacctatggcactgcccgtgaccgccctgctgctgcctctggccctgctgctgcacgcagcccggcctatcctgtggcacgagatgtggcacgagggcctggaggaggccagcaggctgtattttggcgagcgcaacgtgaagggcatgttcgaggtgctggagcctctgcacgccatgatggagagaggcccacagaccctgaaggagacatcctttaaccaggcctatggacgggacctgatggaggcacaggagtggtgcagaaagtacatgaagtctggcaatgtgaaggacctgctgcaggcctgggatctgtactatcacgtgtttcggagaatctccaagggcaaagacacgattccgtggcttgggcatctgctcgttgggctgagtggtgcgtttggtttcatcatcttggtctatctcttgatcaattgcagaaatacaggcccttggctgaaaaaagtgctcaagtgtaatacccccgacccaagcaagttcttctcccagctttcttcagagcatggaggcgatgtgcagaaatggctctcttcaccttttccctcctcaagcttctccccgggagggctggcgcccgagatttcacctcttgaggtacttgaacgagacaaggttacccaacttctccttcaacaggataaggtacccgaacctgcgagccttagctccaaccactctcttacgagctgcttcaccaatcagggatacttctttttccaccttcccgatgcgctggaaatcgaagcttgtcaagtttactttacctatgatccatatagcgaggaagatcccgacgaaggagtcgccggtgcgcccacgggttcctcaccccaacctctccagcctctctcaggagaagatgatgcttattgcacttttcccagtagagacgatctcctcctcttttctccatctcttttggggggaccttcccccccttctacggcacctggcgggtctggtgctggcgaggagcggatgccgccgtccctccaggagcgagtaccacgagattgggatccccagccacttggaccccccacccccggcgtacctgaccttgtcgattttcaacctccccctgaattggtgctgcgagaggctggggaggaagttccggacgctgggccgagggagggcgtgtcctttccatggagtaggcctccaggtcaaggcgagtttagggctctcaacgcgcggctgccgttgaatacagacgcttatctctcactgcaggaactgcaaggtcaggacccaacacatcttgtaggatctggtgctactaatttttctcttttgaagcaagctggagatgttgaagagaaccccggtccggagatgtggcatgagggtctggaagaagcgtctcgactgtactttggtgagcgcaatgtgaagggcatgtttgaagtcctcgaaccccttcatgccatgatggaacgcggaccccagaccttgaaggagacaagttttaaccaagcttacggaagagacctgatggaagcccaggaatggtgcaggaaatacatgaaaagcgggaatgtgaaggacttgctccaagcgtgggacctgtactatcatgtctttaggcgcattagtaagggcagcggcgccaccaacttcagcctgctgaagcaggccggcgacgtggaggagaaccccggccccggtgctggcgcaactggacgcgctatggatggacctcgcttgctgcttcttctgcttctcggggtctctttgggtggtgctaaggaagcatgcccaacgggactttatacgcatagcggagagtgttgcaaagcttgtaacctgggcgaaggcgtcgcgcaaccttgtggtgcaaatcaaaccgtctgcgagccatgtttggactctgttacgtttagtgacgtagtatctgcgacagagccatgcaagccttgtacggaatgtgtaggattgcagagcatgtctgccccttgtgtagaagccgacgatgcagtttgcaggtgcgcgtatggctattaccaagacgaaacaaccggacgatgtgaagcttgccgagtttgtgaagcgggttccgggcttgtattctcctgtcaggataagcagaacaccgtctgcgaagagtgccccgatggtacctacagcgatgaagcgaaccatgtagacccatgcctgccttgcaccgtttgtgaagacacggaacgacagttgcgggaatgtacccggtgggcagacgccgagtgcgaagagattccaggccgctggatcacgcgaagtaccccgccagaaggttccgacagtactgcaccaagcacccaagaaccagaggcgccccccgagcaggacctgattgcctccaccgtggcgggtgttgttactacggttatgggctcatcccagcccgttgttacccgaggaactacagacaacctgattccggtatattgttctatcttggcggctgtagtagttggcttggtcgcgtacatcgctttcaaaagatgaacgccggcgctagtgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgca

SEQ ID NO: 12 - protein, 450 aa, synthetic construct
  REGION 1..450: note = RACCR-beta - FKBP-IL2Rb fusion protein with signal peptide with 2A peptide
MALPVTALLLPLALLLHAARPILWHEMWHEGLEEASRLYFGERNVKGMFEVLEPLHAMMERGPQTLKETSFNQAYGRDLMEAQEWCRKYMKSGNVKDLLQAWDLYYHVFRRISKGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLVGSGATNFSLLKQAGDVEENPG

SEQ ID NO: 13 - protein, 89 aa, synthetic construct
  REGION 1..89: note = FKBP domain
EMWHEGLEEASRLYFGERNVKGMFEVLEPLHAMMERGPQTLKETSFNQAYGRDLMEAQEWCRKYMKSGNVKDLLQAWDLYYHVFRRISK

SEQ ID NO: 14 - protein, 315 aa, synthetic construct
  REGION 1..315: note = IL2Rb domain
GKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 15 - protein, 272 aa, synthetic construct
  REGION 1..272: note = RACCR-gamma - FRB-IL2Rg fusion protein with signal peptide with 2A peptide
MPLGLLWLGLALLGALHAQAGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGEGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPETGSGATNFSLLKQAGDVEENPG

SEQ ID NO: 16 - protein, 108 aa, synthetic construct
  REGION 1..108: note = FRB domain
GVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLGE

SEQ ID NO: 17 - protein, 123 aa, synthetic construct
  REGION 1..123: note = IL2Rg domain
GSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET